(19) United States
(12) Patent Application Publication
(10) Pub. No.: US 2023/0002825 A1
(43) Pub. Date: Jan. 5, 2023

(54) STRUCTURE-BASED DESIGN OF THERAPEUTICS TARGETING RNA HAIRPIN LOOPS

(71) Applicant: The Regents of the University of California, Oakland, CA (US)

(72) Inventors: Feng Guo, Los Angeles, CA (US); Grant Shoffner, Los Angeles, CA (US)

(73) Assignee: The Regents of the University of California, Oakland, CA (US)

(21) Appl. No.: 17/776,943

(22) PCT Filed: Nov. 19, 2020
(86) PCT No.: PCT/US20/61299

§ 371 Date: May 13, 2022

Related U.S. Application Data
(60) Provisional application No. 62/937,657, filed on Nov. 19, 2019.

(51) Int. Cl. C12Q 1/6876

(52) U.S. Cl. CPC C12Q 1/6876

(57) ABSTRACT

The invention provides methods and materials that can be used to determine three dimensional structures of RNA hairpin loops and their complexes with inhibitors easily and quickly. The scaffold RNA, YdaO-type c-di-AMP riboswitch from Thermoanaerobacterpseudethanolicus, readily forms crystals with a large cavity over 60 in diameter. A hairpin of interest can be engineered into the P2 stem of this RNA so that the hairpin is accommodated in the cavity. The fusion RNA is then crystallized, and structures can be determined using X-ray or electron crystallography. Embodiments of the invention can be used to identify compounds that bind hairpin loops in order to, for example, effect therapeutic and other biological activities.

### CROSS REFERENCE TO RELATED APPLICATIONS

This application claims the benefit under 35 U.S.C. Section 119(e) of co-pending and commonly-assigned U.S. Provisional Patent Application Ser. No. 62/937,657 filed on Nov. 19, 2019 and entitled “STRUCTURE-BASED DESIGN OF THERAPEUTICS TARGETING RNA HAIRPIN LOOPS” which application is incorporated by reference herein.

### STATEMENT OF GOVERNMENT SUPPORT

This invention was made with government support under Grant Number 1616265, awarded by the National Science Foundation. The government has certain rights in the invention.

### TECHNICAL FIELD

The invention relates to methods and materials useful to determine three dimensional structures of RNA hairpin loops.

### BACKGROUND OF THE INVENTION

RNA molecules are critical for development of many diseases, such as cancers and RNA viral infections. For this reason, RNA molecules are excellent therapeutic targets. In this context, nearly all RNAs form hairpin secondary structures that are crucial for their function. Consequently, an understanding of these structures is necessary to facilitate the identification and design of therapeutic agents targeting these molecules. However, conventional methods of examining RNAs, such as RNA interference and antisense oligonucleotides, are limited and avoid strong structures.

While conventional technologies can provide some information on RNA structures, the limitations in these technologies make RNA hairpin loops underappreciated targets for therapeutic inhibitor designs.

There is a strong need in this field of technology for new methods and materials useful for obtaining information on the three-dimensional structures of RNA hairpin loops.

### SUMMARY OF THE INVENTION

As described in detail below, we have developed novel scaffold-directed crystallography methods that are useful for obtaining information on the three-dimensional structures of RNA hairpin loops. The RNA crystallization scaffold and associated methods that are disclosed herein can be used to determine three dimensional structures of RNA hairpin loops as well their associations with other agents (e.g. inhibitory agents) easily and quickly. The specific scaffold RNA used in the methods of the invention is the YdaO-type c-di-AMP riboswitch from Thermoanaerobacter pseudethanolicus, an RNA that was discovered to readily form crystals with a large cavity over 60 Å in diameter. As discussed in detail below, we have determined that an RNA of interest can be engineered into the P2 stem of this scaffold RNA so that the hairpin is accommodated in the cavity. The resultant fusion RNA can then be then crystallized, under conditions either similar to or unrelated to that for crystallizing the scaffold alone. The three-dimensional structures of such molecules (e.g. these molecules alone and/or associated with other agents) can then be determined using X-ray or electron crystallography techniques or the like.

The RNA crystallization scaffold and associated methods disclosed herein can be used to identify compounds such as natural and chemically modified oligonucleotides, and small-molecule drugs, that interact with target RNA molecules with high affinity and specificity. This is significant because the interactions between such compounds and RNA hairpin loops can affect biological activities of these molecules in a manner that can modulate their activity in vivo in pathologies such as cancers and RNA viral infections. In addition, because RNAs are involved in nearly every aspect of biology and disease, the methods disclosed herein are widely applicable procedures that can provide information on how to specifically regulate almost any target RNAs. Consequently, the methods disclosed herein allow the observation and assessment of agents such as oligonucleotide analogs that target specific RNAs, including those that function in a wide variety of biological processes such as processes involved in viral replication (e.g. the replication of pathogens such as severe acute respiratory syndrome coronavirus 2, Hepatitis C and Zika), processes involved in pathological conditions such as cancer or neurodegenerative diseases, as well as processes involved in the production of microRNAs for regulating protein-coding genes etc.

The invention disclosed herein has a number of embodiments. One embodiment of the invention is a composition of matter comprising a ribonucleic acid having an at least 90% sequence identity to: GGUUGCCGAAUCCGAAAGGUACGGAGGAACCGCUUUUUGGGGUUAAUC UGCAGUGAAGCUGCAGUAGGGAUACCUUCUGUCCCGCACCCGACAGCUA ACUCCGGAGGCAAUAAAGGAAGGAG (SEQ ID NO: 1). Typically, the polynucleotide comprises the sequence of SEQ ID NO: 1. In this composition, residues 14-17 of SEQ ID NO: 1 (GAAA) of the ribonucleic are replaced with a heterologous segment of nucleic acids that is between 4 and 33 nucleotides in length (the at least 90% sequence identity noted above does not include the heterologous segments of nucleic acids that can be inserted in to this ribonucleic acid at residues 14-17). In these compositions, the heterologous segment of nucleic acids is typically one that forms a loop structure in a naturally occurring RNA molecule. In certain embodiments of the invention, the heterologous segment of nucleic acids includes a complete loop structure, and optionally between 0-5 base pairs of a stem structure in the naturally occurring RNA molecule. Optionally these compositions can further comprise an agent that binds to the ribonucleic acid, for example a polynucleotide that hybridizes to the ribonucleic acid.

Another embodiment of the invention is a system or kit for observing RNA structures comprising a plasmid comprising a DNA sequence encoding a ribonucleic acid having an at least 90% (and optionally less than 100%) identity to: GGUUGCCGAAUCCGAAAGGUACGGAGGAACCGCUUUUUGGGGUUAAUC UGCAGUGAAGCUGCAGUAGGGAUACCUUCUGUCCCGCACCCGACAGCUA ACUCCGGAGGCAAUAAAGGAAGGAG (SEQ ID NO: 1). In certain embodiments, the plasmid further comprises a promoter for expressing or transcribing the ribonucleic acid, and/or the system or kit further comprises an RNA polymerase. Optionally the system or kit further comprises one or more primers that hybridize to a stretch of nucleic acids in the plasmid.

Yet another embodiment of the invention is a method of obtaining information on a structure of a ribonucleic acid. This method comprises substituting residues 14-17 (GAAA) of SEQ ID NO: 1 (or a ribonucleic acid having an at least 90% to SEQ ID NO: 1) with a heterologous segment of nucleic acids that is between 4 and 33 nucleotides in length to so as to form a fusion ribonucleic acid molecule, crystallizing the fusion RNA, performing an X-ray or electron crystallographic technique on the fusion ribonucleic acid molecule, and then observing the results (e.g. electron density maps of a X-ray or electron crystallographic technique) to obtain information on the three-dimensional structure of the heterologous segment of nucleic acids. In certain embodiments of these methods, the fusion ribonucleic acid molecule is combined with an agent that binds to the ribonucleic acid prior to the crystallographic analysis (e.g. a polynucleotide that hybridizes to the ribonucleic acid) so that the structure of the RNA/agent complex can be observed. Typically in these methods, the crystallographic analysis includes a comparison to a control sample lacking the agent that binds to the ribonucleic acid. Optionally in these methods, a plurality of fusion ribonucleic acid molecules are combined with a plurality of agents that bind to the ribonucleic acid (e.g. in high throughput screening) prior to the X-ray or electron crystallographic technique. In some embodiments of the invention, at least two agents are combined with the fusion ribonucleic acid molecules.

In illustrative working embodiments of the invention, we examined nine structures of pri-miRNA hairpin loops. These studies determined that loops 4-8 nucleotides in length are more structured than previously thought, making these and moderately longer loops excellent targets for therapeutic agents. In embodiments of the invention, a target loop does not have to be of particular length, and can be longer or shorter than the available examples. This realization and our novel structural determination methods allow artisan to identify lead oligonucleotide compounds and go through iterative rounds of structure-based refinement quickly and cost effectively. The methods of the invention have broad applications because they target processes that are important for fighting infectious diseases and cancers, age related pathologies and neurodegenerative diseases, as well as genetic disorders such as the DiGeorge syndrome and the like.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

Brief descriptions of the drawing are found in the text below.

FIGS. 1A-IE. Analysis of pri-miRNA terminal loops and search for potential crystallization scaffolds. FIG. 1(a): shows the distribution of pri-miRNA apical loop lengths. FIG. 1(b): shows a comparison of the largest spherical cavity (with radius Rₘₐₓ) present in each RNA crystal structure against the diffraction resolution of the structure. Crystal forms with a single molecule in the asymmetric unit are shown as green crosses, and all others as black dots. FIG. 1(c): shows a structure of RNA. FIG. 1(d): shows a secondary structure of the YdaO-type ci-di-AMP riboswitch. FIG. 1(e): shows a crystal packing of the riboswitch (PDB ID 4QK8). Molecules surrounding a large central channel (parallel to the c-axis) are colored grey, and a blue sphere with a radius of 31 Å is positioned in the channel to illustrate its size. The L2 stem loops terminating inside the channel are green. FIG. 1(f): shows a native gel analysis of W.T. YdaO and fusions with the pri-miR-9-1 terminal loop with 0-3 base pairs from the stem.

FIGS. 2A-2F. Atomic structures of pri-miRNA terminal loops 8-6 nt in length determined by scaffold-directed crystallography. Throughout the figure, the last base pair from the scaffold P2 stem is colored grey. FIGS. 2A and 2D-2F are shown in stereographic view. Inset shows the secondary structure of the loop. The 2Fo-Fc electron density map is contoured at the level shown in each panel. FIG. 2(a) shows pri-miR-378a (378a+0 bp). FIG. 2(b) shows pri-miR-378a loop with one base pair from the stem (378a+1 bp). FIG. 2(c) shows the 378a+1 bp structure and electron density. FIG. 2(d) shows pri-miR-340 (340+1 bp). FIG. 2(e) shows pri-miR-300 (300+0 bp). The neighboring canonical pair in pri-miR-300 is C-G, identical to the pair in the scaffold. Therefore, the structure is essential 300+1 bp. FIG. 2(f) shows pri-miR-202 (202+1 bp).

FIGS. 3A-3D. Structures of shorter (4-5 nt) pri-miRNA loops. Color scheme is identical to that in FIG. 2. FIG. 3(a) shows pri-miR-208a (208a+1 bp). FIG. 3(b) shows pri-miR-320b-2 (320b-2+1 bp). FIG. 3 (c) shows pri-miR-449c (449c+1 bp). FIG. 3(d) shows pri-miR-19b-2 (19b-2+1 bp).

FIGS. 4A-4E. Structural consensus, non-canonical pairs, and asymmetric flexibility of human pri-miRNA apical junctions and loops. FIG. 4(a) shows a structural alignment of all eight loops shown in FIGS. 2 and 3. Positions that align well among most or all of the structures are labeled. FIG. 4(b) shows a plot of folding AG values of the eight pri-miRNA apical junctions and loops measured with 50 mM NaCl. Error bars represent standard deviations, obtained from 4-6 repeats. Each RNA contains the apical loop and the immediately neighboring base pair from the stem, along with five common base pairs (see FIG. 8a for RNA secondary structures and Table 2 for detailed thermodynamic parameters). FIG. 4(c) shows observed and expected counts of human pri-miRNAs with the indicated apical loop-closing residue pairs. The expected counts are estimated based on the abundance of 5′ and 3′ loop residues. FIG. 4(d) shows the average atomic displacement parameter (ADP) per residue, with all loops plotted on the same scale. The 5′ and 3′ end represent the terminal base pair of the pri-miRNA stem loop. Structure drawings illustrating ADP distribution are presented in FIG. 10. FIG. 4(e) shows the root-mean-square fluctuations (RMSF, Å) determined for each residue by molecular dynamics. Symbols and coloring are identical to those in FIG. 4(d).

FIGS. 5A-5K. Association of the DGCR8 Rhed domain with pri-miRNA apical junctions. FIGS. 5(a)-5(h) Quantification of gel shift assays, with representative gel images shown in FIG. 11. Data points represent the mean fraction bound±standard error (SE) from three replicate experiments. Data were fit with the Hill equation and the dissociation constant (K_{d}) are shown (±SE). FIG. 5(i) shows a comparison of the free energy of Rhed binding (RTln(K_{d})) to the length of the terminal loop, as predicted by mfold. FIG. 5(j) shows the same as FIG. 5(i) except that the loop lengths are adjusted with bases involved in non-canonical pairs excluded.

FIGS. 6A-6C. Results from a systematic mutagenesis of the U-U pair we observed in several crystal structures of pri-miRNA apical junctions (U-U pairs are among the best processed pri-miRNA variants). Terminal residues in pri-miRNA apical loops fine-tune miRNA production. FIG. 6(A) shows a schematic of dual-pri-miRNA constructs for measuring miRNA maturation efficiency in mammalian cells. Each pri-miRNA fragment contains the hairpin and about 30-nt flanking sequence on each side, totaling ˜150 nt. The pri-miR-9-1 fragment is unchanged and is used for normalization. The terminal loop residues of the 3′ pri-miRNA fragment are subjected to mutagenesis. The abundance of both mature miRNAs is measured using quantitative RT-PCR. FIG. 6(B) shows a maturation efficiencies of pri-miR-340 variants (miR-340/miR-9 ratios). FIG. 6(C) shows maturation efficiencies of pri-miR-193b variants. In these scatter plots, individual data points are shown as gray dots. The bars indicate means and standard deviations.

FIGS. 7A-7I. Simulated annealing composite omit maps calculated for all pri-miRNA loops. Color scheme is the same as FIGS. 2 and 3. All maps are contoured to 1.1σ. See the Methods section for details on calculation of individual maps. FIG. 7A shows 378a+0 bp. FIG. 7B shows 378a+1 bp. FIG. 7C shows 340+1 bp. FIG. 7D shows 300+0 bp. FIG. 7E shows 202+1 bp. FIG. 7F shows 208+1 bp. FIG. 7G shows 449c+1 bp. FIG. 7H shows 320b-2+1 bp. FIG. 7I shows 19b-2+1 bp.

FIGS. 8A-81. RNA constructs used for melting and binding assays. FIG. 8(a) shows short RNA oligos used for optical melting assays. A common 5-bp helical segment was used as the stem for all hairpins (grey base pairs). The pri-miRNA apical junction and loop nucleotides are black. FIGS. 8(b)-8(i) show secondary structure predictions for all pri-miRNA fragments used in the Rhed binding assay. Additional G-C pairs added to the base of the stem to enhance transcription are highlighted in yellow. The box shows the sequence of apical loop and terminal base pair of the stem used to determine crystal structures.

FIGS. 9A-9B. Comparison of pri-miRNA terminal loop structures to similar RNA folds found in the PDB. FIG. 9A shows a cartoon representation of the 8-nt loop of pri-miR-378a (378a+1, left), FIG. 9B shows similar loops from distinct structures of RNase P(2), guanidine-I riboswitch(3), and tRNA^{Phc} (4).

FIGS. 10A-10H. Estimating the flexibility of the apical loop with atomic displacement parameters (ADPs). Each structure shown in FIG. 10(a)-FIG. 10(h) is colored with the lowest ADPs in blue to the highest in red. The insets show the range of ADP plotted.

FIG. 11A-11H. Example gel shift assays for each pri-miRNA fragment binding to the Rhed. The pre miRNA fragment is identified above each gel, and the free RNA and protein-bound species are labeled in the gels. Rhed dimer concentrations (μM) used in the binding reactions are shown below the gels.

FIGS. 12A-12b. Analysis of the pri-miR-223 apical loop sequencing data from the previously reported high-throughput mutagenesis and processing assay(5). FIG. 12(a) shows a predicted secondary structure of the upper region of the pri-miR-223 hairpin. Base coloring reflects the level of evolutionary conservation in the Rfam entry for this RNA (Rfam accession: RF0064). The major miRNA product from the 3p arm is highlighted in blue. Red letters show mutations relative to the WT sequence. Compared to alternative secondary structure shown in the inset, this model is likely to the dominating conformation because it produces an optimal upper stem length of ˜23-bp above the Drosha cleavage sites and places evolutionary conserved residues inside the stem and less conserved position in a bulge loop. FIG. 12(b) shows a heatmap showing the frequency of C-A pairs in the 9-nt pri-miR-223 loop sequencing data. The lower left diagonal matrix shows the percent frequency in the input library and the upper right diagonal matrix shows the frequency in the processed RNA. For reference, the wild-type loop sequence is shown along the diagonal. C-A pair is enriched to 69% in the processed faction versus 22% in the input.

FIG. 13. NMR ensemble for pri-miR-20b, showing the U-G pair at the apical junction and stacking of the neighboring 5′ G residue (6).

FIGS. 14A and 14B. RNA structures. FIG. 14A shows the secondary structure of HCV cis-acting replication element; and FIG. 14B shows the HCV IRES domain IIIb (see, e.g. Quade et al., Nature Communications volume 6, Article number: 7646 (2015)).

### DETAILED DESCRIPTION OF THE INVENTION

Many of the techniques and procedures described or referenced herein are well understood and commonly employed using conventional methodology by those skilled in the art. In the description of the preferred embodiment, reference may be made to the accompanying drawings which form a part hereof, and in which is shown by way of illustration a specific embodiment in which the invention may be practiced. It is to be understood that other embodiments may be utilized and structural changes may be made without departing from the scope of the present invention.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

Metazoan pri-miRNAs fold into characteristic hairpin structures that are recognized by the Microprocessor complex during processing. Essential for this recognition, the apical junction that joins the hairpin stem and loop directs the DGCR8 RNA-binding heme domain (Rhed) to the apex of the hairpin. Here we describe a scaffold-directed crystallography method and report the structures of numerous human pri-miRNA apical junctions and loops. These structures reveal a consensus in which a non-canonical base pair and at least one 5′ loop residue stack on top of the hairpin stem. The non-canonical pairs contribute to thermodynamic stability in solution. U-U and G-A pairs are highly enriched at the apical junctions of human pri-miRNAs. We also find that the Rhed binds longer loops more tightly, biochemically explaining why pri-miRNAs with shorter loops are often poorly processed. Our disclosure provides a structural basis for understanding pri-miRNAs and relevant molecular mechanisms of microRNA maturation.

As discussed below, we have developed methods and materials that are useful to determine three-dimensional structures of pri-miRNA apical junctions and loops for their important roles in miRNA maturation and regulation (7-10). These moieties are present in both pri-miRNAs and pre-miRNAs and thereby their structures affect both Drosha and Dicer cleavage steps (8). The apical junctions and loops are also targets for drug discovery (11). To date only two pri-miRNA apical stem-loops have been structurally characterized in ligand-free states, using NMR spectroscopy (6, 11, 12). The 13-nt pre-miR-20b apical loop folds to well-defined rigid structures (6), whereas weak signals suggest that the 14-nt pri-miR-21 loop is unstructured (11, 12). The human genome encodes 1,881 pri-miRNA hairpins that differ from each other greatly (13). Toward surveying the large number of pri-miRNA structures, we have developed a scaffold-directed crystallization technique that enables rapid determination of hairpin loop structures without interference from the crystal lattice. We report nine apical junction and loop structures from eight pri-miRNAs and biochemical characterization of their interactions with Rhed.

Embodiments of the invention include compositions of matter comprising a ribonucleic acid having an at least 90% sequence identity to: GGUUGCCGAAUCCGAAAGGUACGGAGGAACCGCUUUUUGGGGUUAAUC UGCAGUGAAGCUGCAGUAGGGAUACCUUCUGUCCCGCACCCGACAGCUA ACUCCGGAGGCAAUAAAGGAAGGAG (SEQ ID NO: 1). Embodiments of the invention preferably exhibit at least about a 91%, 92%, 93%, 94%, 95%, 96%, 97%, 98%, or 99% identity to the polynucleotide sequence of SEQ ID NO: 1. The percent identity may be readily determined by comparing sequences of polynucleotide variants with the corresponding portion of a full-length polynucleotide of SEQ ID NO: 1 (wherein the sequence identity noted above does not include the heterologous segments of nucleic acids that can be inserted in to this ribonucleic acid in place of residues 14-17). Some techniques for sequence comparison include using computer algorithms well known to those having ordinary skill in the art, such as Align or the BLAST algorithm (Altschul, J. Mol. Biol. 219:555-565, 1991; Henikoff and Henikoff. PNAS USA 89:10915-10919, 1992)). Default parameters may be used.

Typically, the polynucleotide comprises the sequence of SEQ ID NO: 1. In this composition, residues 14-17 of SEQ ID NO: 1 (GAAA) of the ribonucleic are replaced with a heterologous segment of nucleic acids that is between 4 and 33 nucleotides in length (the at least 90% sequence identity noted above does not include the heterologous segments of nucleic acids that can be inserted in to this ribonucleic acid at residues 14-17). In one illustrative embodiment, the polynucleotide comprises GGUUGCCGAAUCCXGGUACGGAGGAACCGCUUUUUGGGGUUAAUCUGC AGUGAAGCUGCAGUAGGGAUACCUUCUGUCCCGCACCCGACAGCUAACU CCGGAGGCAAUAAAGGAAGGAG (SEQ ID NO: 29), wherein X comprises between 4 and 33 heterologous nucleotides (e.g. those comprising a three-dimensional structure in a naturally occurring RNA molecule such as a human miRNA) selected from A, U, G and C. In these compositions, the heterologous segment of nucleic acids is typically one that forms a three-dimensional structure in a naturally occurring RNA molecule (e.g. a loop structure). In certain embodiments of the invention, the heterologous segment of nucleic acids includes a complete loop structure, and optionally between 0-5 base pairs of a stem structure in the naturally occurring RNA molecule. Optionally these compositions can further comprise an agent that binds to the ribonucleic acid, for example a polynucleotide that hybridizes to the ribonucleic acid.

Another embodiment of the invention is a system or kit for observing RNA structures comprising one or more plasmids comprising a DNA sequence encoding a ribonucleic acid having an at least 90% (and optionally less than 100%) identity to: GGUUGCCGAAUCCGAAAGGUACGGAGGAACCGCUUUUUGGGGUUAAUC UGCAGUGAAGCUGCAGUAGGGAUACCUUCUGUCCCGCACCCGACAGCUA ACUCCGGAGGCAAUAAAGGAAGGAG (SEQ ID NO. 1). In some embodiments of the invention, the one or more plasmids comprise a polynucleotide sequence having an at least 90% identity the sequence GGTTGCCGAATCC (SEQ ID NO: 27) and/or a polynucleotide sequence having an at least 90% identity the sequence GGTACGGAGGAACCGCTITMGGGGTTAATCTGCAGTGAAGCTGCAGTAG GGATACCTTCTGTCCCGCACCCGACAGCTAACTCCGGAGGCAATAAAGGA AGGAG (SEQ ID NO: 28). In certain embodiments, the one or more plasmids further comprises a promoter for expressing or transcribing the ribonucleic acid, and/or the system or kit further comprises an RNA polymerase. Optionally the system or kit further comprises one or more primers that hybridize to a stretch of nucleic acids in the plasmid.

Yet another embodiment of the invention is a method of obtaining information on a structure of a ribonucleic acid. This method comprises substituting residues homologous to residues 14-17 (GAAA) of SEQ ID NO: 1 (or a ribonucleic acid having an at least 90% to SEQ ID NO: 1) with a heterologous segment of nucleic acids that is between 4 and 33 nucleotides in length (e.g. a heterologous segment that is 4, 5, 6, or 7 nucleotides etc., up to 33 nucleotides in length) to so as to form a fusion ribonucleic acid molecule, crystallizing the fusion RNA, performing structural analysis such as one comprising an X-ray or electron crystallographic technique on the crystallized fusion ribonucleic acid molecule, and then observing the results so as to obtain information on the three-dimensional structure of the heterologous segment of nucleic acids. In certain embodiments of these methods, the fusion ribonucleic acid molecule is combined with an agent that binds to the ribonucleic acid prior to the crystallographic analysis (e.g. a polynucleotide or other agent that binds to the heterologous segment of the ribonucleic acid) so that the structure of the RNA/agent complex can be observed. Typically in these methods, the crystallographic analysis includes a comparison to a control sample lacking the agent that binds to the ribonucleic acid. Optionally in these methods, a plurality of fusion ribonucleic acid molecules are combined with a plurality of agents that bind to the ribonucleic acid (e.g. in a high throughput screening procedure) prior to the structural analysis (e.g. X-ray or electron crystallographic) technique. In some embodiments of the invention, at least two agents are combined with the fusion ribonucleic acid molecules.

A related embodiment of the invention includes methods of performing a crystallographic analysis on a polynucleotide. Typically these methods comprise: selecting a first polynucleotide, wherein the first polynucleotide comprises a polynucleotide sequence of a first miRNA; identifying a segment of polynucleotides that forms a first loop region in the first miRNA; selecting a second polynucleotide, wherein the second polynucleotide comprises the polynucleotide sequence of a second miRNA; identifying a segment of polynucleotides that forms a first loop region in the second miRNA; forming a fusion polynucleotide selected so that the segment of polynucleotides comprising the first loop region on the first polynucleotide is substituted or swapped with the segment of polynucleotides comprising the first loop region on the second polynucleotide; and then crystallographically analyzing the fusion polynucleotide so as to observe a three dimensional structure of the fusion polynucleotide; so that a crystallographic analysis of the polynucleotide is performed. In certain embodiments of these methods, the first miRNA is a miRNA having at least 90% sequence identity to: GGUUGCCGAAUCCGAAAGGUACGGAGGAACCGCUUUUUGGGGUUAAUC UGCAGUGAAGCUGCAGUAGGGAUACCUUCUGUCCCGCACCCGACAGCUA ACUCCGGAGGCAAUAAAGGAAGGAG (SEQ ID NO: 1), wherein: residues 14-17 (GAAA) of the ribonucleic acid are replaced with a heterologous segment of nucleic acids comprising the first loop region on the second polynucleotide that is between 4 and 33 nucleotides in length. In certain embodiments of the invention, the first polynucleotide comprises the sequence of SEQ ID NO: 1; and/or the second miRNA comprises a human miRNA. Typically in these methods, the crystallographic analysis is an X-ray or electron crystallographic technique; and/or the crystallographic analysis is performed in the presence of agent that binds to the fusion polynucleotide (e.g. an antisense oligonucleotide having homology to a segment of nucleic acids comprising a first loop region on the second polynucleotide).

In illustrative working embodiments of the invention, we examined nine structures of pri-miRNA hairpin loops. These studies determined that loops 4-8 nucleotides in length are more structured than previously thought, making these and moderately longer loops excellent targets for therapeutic agents. In embodiments of the invention, a target loop does not have to be of particular length, and can be longer or shorter than the available examples. This realization and our novel structural determination methods allow artisan to identify lead oligonucleotide compounds and go through iterative rounds of structure-based refinement quickly and cost effectively. The methods of the invention have broad applications because they target processes that are important for fighting infectious diseases such as Coronavirus disease 2019, as well as cancers, age related pathologies and neurodegenerative diseases, and genetic disorders such as Duchenne muscular dystrophy, the DiGeorge syndrome and the like. In one illustration of this, embodiments of the invention can be used to test and examine new antisense therapeutics that are designed to target genes that are associated with the pathogenesis of human cancers, especially those cancers that are not amenable to small-molecule or antibody inhibition.

As discussed below, we determined the three-dimensional structures of human primary transcripts of microRNAs (pri-miRNAs) (1). Briefly, pri-miRNAs are recognized and cleaved in the nucleus by the Microprocessor complex that contains the Drosha ribonuclease and its RNA-binding partner protein DGCR8. Pri-miRNA apical junctions and loops are also the binding sites for other RNA-binding proteins and metabolites that regulate microRNA maturation. More importantly, such pri-miRNA apical loops can then be observed when targeted by agents such as polynucleotides, small-molecules, and the like. In this way, mature, functional microRNAs and their structures can be observed when bound to or otherwise modulated by agents that, for example, have therapeutic potential.

Further aspects and embodiments of the invention are discussed in the following sections.

### Survey of Pri-miRNA Apical Loop Length

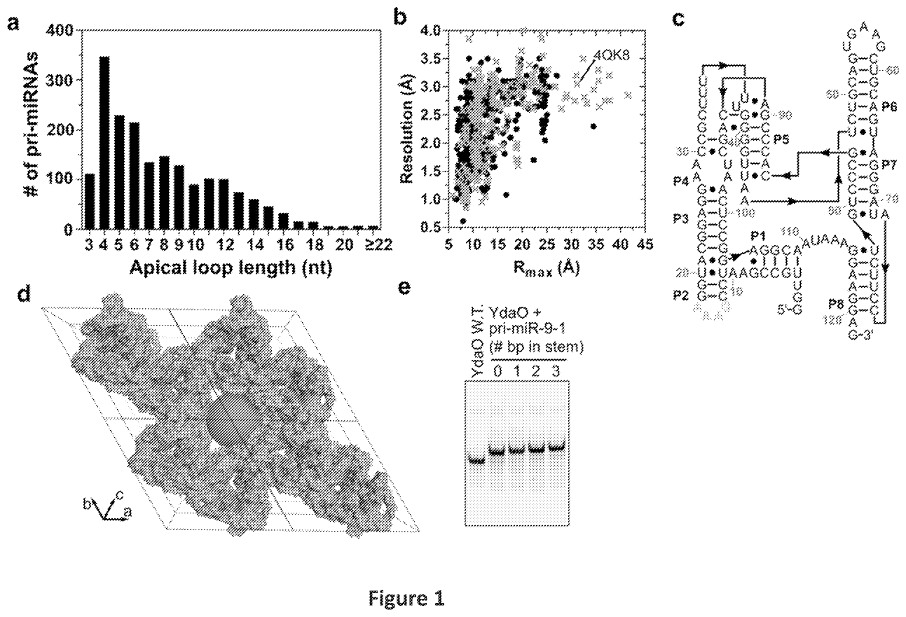

A previous investigation showed that pri-miRNAs with short (<10 nt) apical loops tend to be processed inefficiently by Microprocessor (7). Considering and building upon this, we compiled a list of human pri-miRNA apical loop sequences based on predicted secondary structures we produced using mfold (14) and similar ones provided by miRBase (13). Majority of them (1,314 out of 1,881, 70%) are less than 10 nt long, with the highest frequencies in the 4-6 nt range (FIG. 1a). RNA secondary structure prediction programs tend to include base pairs in relatively long loops that are not necessarily stable (6, 11). We partially addressed this apparent bias by disregarding 1 or 2 base pairs that are isolated from the hairpin stem. Although the list might still underestimate the number of longer loops, it nevertheless reflects the best of our knowledge. Therefore, for most pri-miRNA recognition events, the Rhed must interact with a relatively short apical loop in order to access the apical junction.

### Scaffold-Directed Crystallography

To determine the three-dimensional structures of pri-miRNA apical junctions and loops, we developed a scaffold-directed crystallization approach. The concept is to fuse the target (unknown) sequence onto a scaffold molecule known to crystallize well and with a crystal structure available. The fusion should crystallize under conditions similar to that for the scaffold alone. The crystal lattice should be able to accommodate the target moiety. The scaffold structure allows the structure of the fusion to be determined via molecular replacement.

To identify a suitable scaffold, we mined the Protein Data Bank for RNA crystals fulfilling four criteria. For each RNA structure entry, we first identified the largest sphere that can be accommodated in the lattice cavity, as characterized by the radius Rₘₐₓ (FIG. 1b). We considered the diffraction resolution reported. To simplify the design, we limited the search to entries with one molecule in the asymmetric unit. Finally, we manually reviewed the crystal lattices to find stem-loops that point toward the lattice cavity so that an NA hairpin can be fused to. Amongst hundreds of structures surveyed, we identified only one RNA meeting these requirements, the YdaO-type c-di-AMP riboswitch from Thermoanaerobacter pseudethanolicus (abbreviated from here on as YdaO) (15).

The YdaO crystal lattice contains large solvent channels (Rₘₐₓ≈30 Å) with the short P2 stem positioned inside the channel and away from neighboring molecules (FIG. 1c,d). The riboswitch has a complex pseudo-two-fold symmetric ‘cloverleaf’ fold (FIG. 1d). We replaced the GAAA tetraloop on the YdaO P2 stem with the 14-nt pri-miR-9-1 apical loop plus 0-3 additional base-pairs from the stem. All four fusion RNAs, after annealing in the presence of the c-di-AMP ligand, migrated as single bands on a native gel (FIG. 1e), indicating that the engineered pri-mi-RNA sequences do not interfere with the scaffold folding.

For our representative set of short pri-miRNA loops, we generated fusions with the YdaO scaffold containing the loop plus a various number of base pairs from the stem, and screened for crystallization. We succeeded in obtaining crystals for constructs containing 0 or 1 base pair from the pri-miRNA stems. These crystals belong to the same space group, P3121, with similar cell dimensions (Table 1). We collected X-ray diffraction data and determined their structures with resolution ranging from 2.71 to 3.08 Å (Table 1). For three pri-miRNAs, we also collected single-wavelength anomalous dispersion (SAD) data with redundancy in the 79-115 range. These SAD data contributed to phasing and refinement. The refined native structures showed that the scaffold moieties are very similar to that of the wild type (WT), with C1′ root-mean-square deviation (RMSD) values ranging from 0.22 to 1.18 Å. Below we describe the pri-miRNA moieties. Unlike most RNA loop structures in the PDB, our structures are free from crystal contacts and interactions with ligands, and thereby reflect their own folding propensities.

### Structures of Pri-miRNA Apical Junctions and Loops

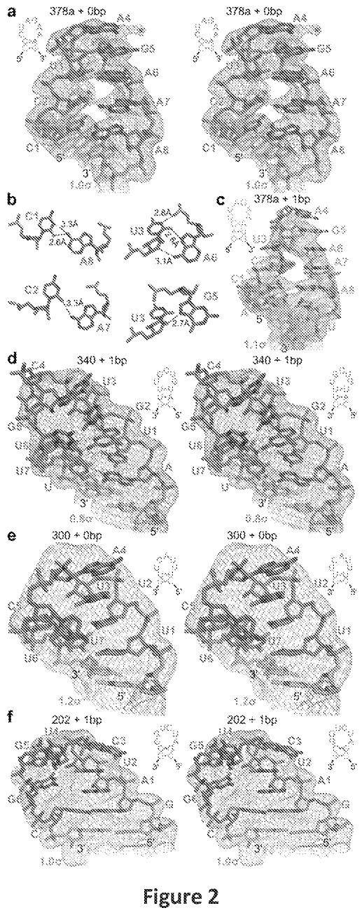

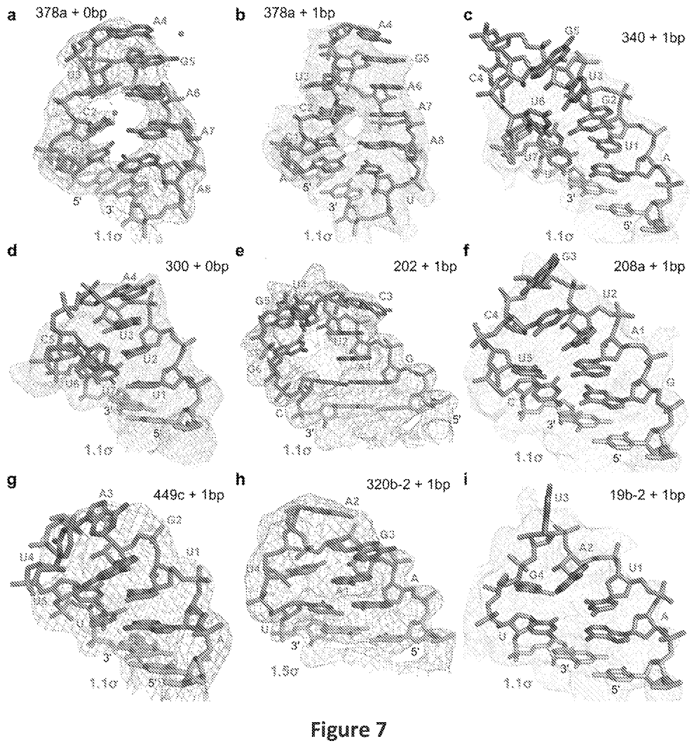

Our series of pri-miRNA loop structures cover the most frequent loop lengths in humans, ranging from 4 to 8 nt. The longest loop was 8 nt, from pri-miR-378a (termed 378a+0 bp, FIG. 2a and FIG. 7a). As RNA loops can be flexible, they are often not well resolved in the electron density. To our surprise, the 2Fₒ-F_{c} map of 378a+0 bp revealed a highly structured conformation with clear density for all residues. The 378a+0 bp structure clearly shows that outermost residues of the loop, C1 and A8, form a non-canonical pair, which creates a platform onto which bases from the remainder of the loop stack (FIG. 2b). On the 5′ end, C2 and U3 stack above C1. From the 3′ side, A4, G5, A6, and A7 stack in four layers above A8. Across the two stacks of bases, hydrogen bonds between C2^{O2}-A7^{N6} (3.3 Å) U3^{O4′}-A6^{N6} (3.1 Å), U3^{N3}-A6^{OP2} (2.8 Å), U3^{O2}-A6^{N7} (2.6 Å), and U3^{2′OH}-G5^{N7} (2.7 Å) further stabilize the loop (FIG. 2b). Every loop nucleotide of pri-miR-378a is coordinated by H-bonding except A4.

We also solved the structure of the pri-miR-378a apical loop with one base pair from the stem (378a+1 bp, FIG. 2c and FIG. 7b). The models for both loops are in close agreement (1.4 Å RMSD over all non-hydrogen atoms in the loop, FIG. 2c). The 378a+1 bp structure confirms the non-canonical C1-A8 pair. Interestingly, the fact that 378a+0 bp and 378a+1 bp are nearly identical suggests that the loop conformation is not strongly influenced by the terminal A:U pair from the pri-miRNA stem.

The structures of pri-miR-340 (340+1 bp) and pri-miR-300 (300+0 bp) contain 7-nt loops. The 340+1 bp structure confirms the presence of the terminal A-U pair, which is capped by an unexpected U1-U7 pair (FIG. 2d and FIG. 7c). The G2 and U3 bases from the 5′ end of the loop stack on top of the U-U pair. This leaves just three residues (C4, G5, and U6) in a more flexible conformation at the top of the loop. In the 300+0 bp structure, the terminal C-G pair of the scaffold is identical to the last base pair of the pri-miR-300 stem, thus this structure is effectively 300+1 bp. As in the case of 378a and 340, we observe a non-canonical pairing between U1 and U7 (FIG. 2e and FIG. 7d). Likewise, a chain of base-stacking interactions between U1, U2, U3 and A4 orders the 5′ end of the loop. U6 is within hydrogen bonding distance with the U2 base, almost forming another non-canonical pair. C5 is outside the density and appears to be more flexible.

In the structure of pri-miR-202 (6-nt loop), we did not observe non-canonical base pairs. However, similar to other structures, the A1 base at the 5′ end of the loop stacks to the final G-C pair of the pri-miRNA stem (FIG. 2f and FIG. 7e). The rest of the loop shows continuous electron density at 1σ, but we could not determine the conformation with high confidence. Overall, the structures of the relatively long (6-8-nt) pri-miRNA loops reveal extensive base stacking and non-canonical base pairing interactions, perhaps stabilizing the loops more than previously anticipated. As a consequence, fewer loop residues is conformationally flexible.

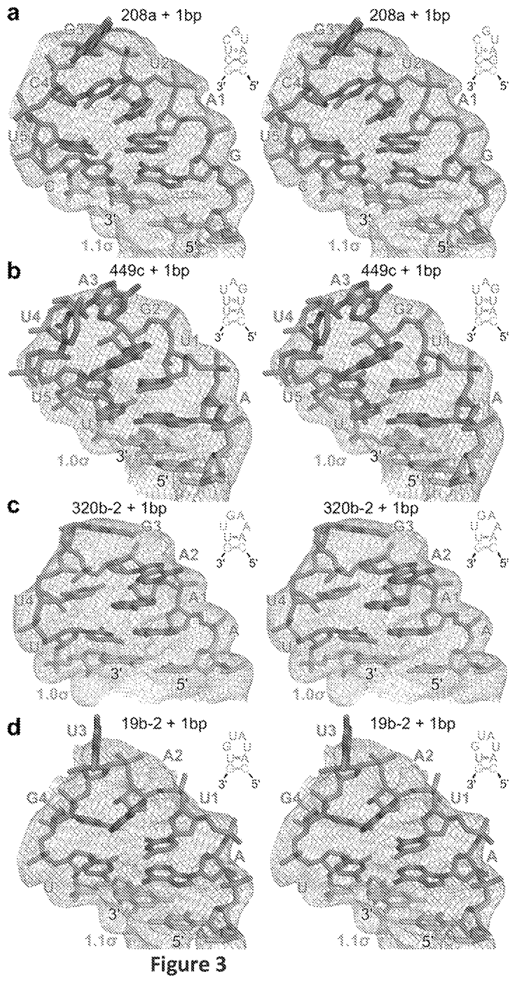

Next, we investigated the structures of shorter pri-miRNA terminal loops (4-5 nt. FIG. 3). The structure of pri-miR-208a (208a+1 bp) with a 5-nt loop revealed an unpredicted A1-U5 Hoogsteen pair positioned above the final G-C pair from the stem (FIG. 3a and FIG. 7f). The central 3 nt of the loop, U2, C4, and G3 base-stack together and onto the A1 base in the Hoogsteen pair. In addition, the non-canonical U-U pair from 340+1 bp is recapitulated between U1 and U5 in the structure of pri-miR-449c (FIG. 3b and FIG. 7g). Positions U1 and G2 stack together above the terminal base pair, leaving just A3 and U4 outside the density. The two pentaloops share a theme: the two outermost residues form non-canonical base pairs, whereas the central three residues are unpaired and some of their bases are stacked.

Similar to the structure of 202+1 bp above, for pri-miR-320b-2 (5-nt loop), the A1 residue of the loop sits atop the terminal A-U pair of the stem (FIG. 3c and FIG. 7h). Finally, in the tetraloop structure of pri-miR-19b-2 (19b-2+1 bp) the 5′ loop nucleotide U1 stacks above the terminal base pair and a partial stacking interaction of A2 on top of U1 (FIG. 3d and FIG. 7i). U3 and G4 are mostly outside the electron density, although there may be a contact between G4^{N7} and the 2′-OH of A2 (˜2.6 Å). These structures confirm that the non-canonical pairing and base stacking of the 5′ loop residues witnessed in longer loop structures also dominate the folding of the shorter loops.

### A Structural Consensus of Pri-miRNA Apical Junctions

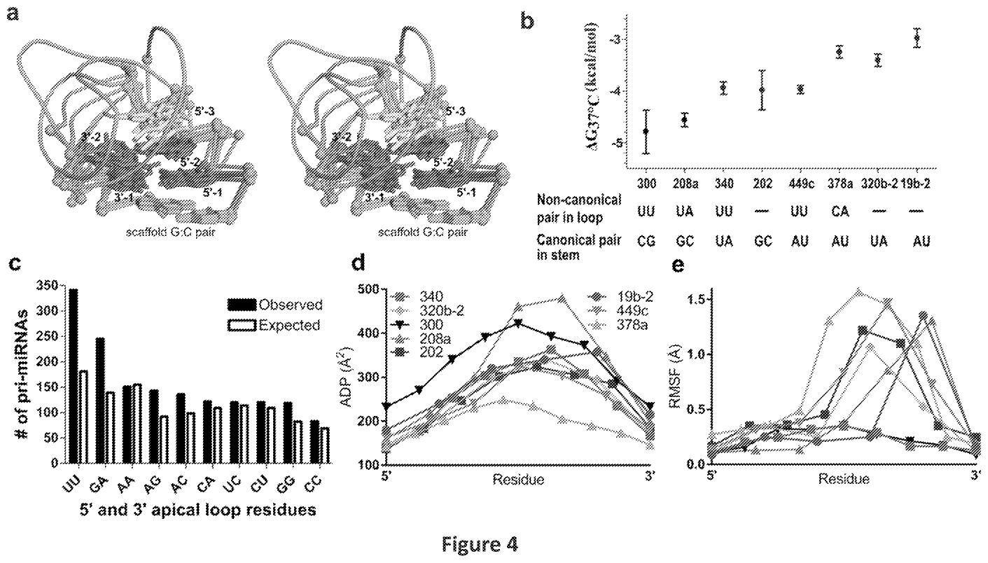

Our pri-miRNA stem-loop structures point toward a common set of structural features defining the terminal loop. To further illustrate these features, we generated a structural alignment of all eight pri-miRNA loops (FIG. 4a). First, we always observe the mfold-predicted canonical base pair at the apical end of the pri-miRNA stem (5′-1 paired with 3′-1). Because the loops are of different sizes, here we use 5′-1 to represent the first residue from the 5′-end of the pri-miRNA sequence, and 3′-1 to represent the first residue from the 3′-end. Second, in all structures the first nucleotide on the 5′ end of the loop base-stacks with the terminal base pair (5′-2 stacking with 5′-1/3′-1). Third, in five of the eight loops (378a, 340, 300, 208a, 449c), this base stacking is also accompanied by a non-canonical base pair (5′-2 pairing with 3′-2), effectively making the apical loop shorter than predicted by two nucleotides. Fourth, all eight structures reveal at least one additional level of base-stacking interactions on the 5′ side (5′-3 stacked on 5′-2). In contrast, only two structures indicate second-layer stacking on the 3′ side. Beyond these common features, other residues of the pri-miRNA loops appear to adopt quite different conformations or are flexible.

### Non-Canonical Base Pairs Contribute to Thermodynamic Stability

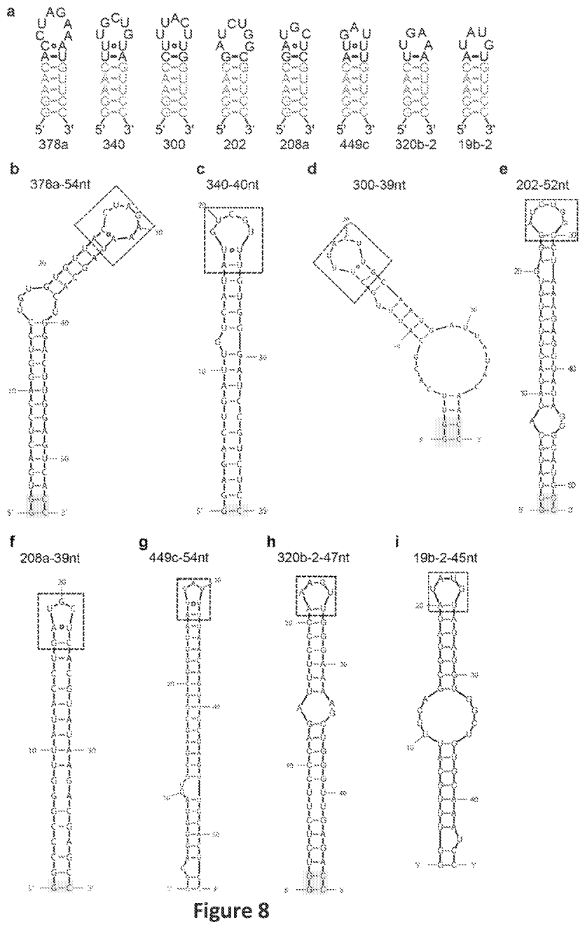

To test if the structures of apical junctions and loops we observed contribute to their stability in solution, we fused the eight pri-miRNA sequences to a common 5-bp helical segment (FIG. 8a) and measured their thermodynamic parameters using optical melting. Like in the crystal structure, each pri-miRNA sequence contains the apical loop and an immediately neighboring canonical base pair from the stem so that a minimal apical junction is included. We expect that the canonical stem base pairs contribute differentially to the overall stability, with the G-C or C-G pairs in three pri-miRNAs being more stable than the A-U and U-A pairs in the others. However, this difference does not fully explain the free energy changes (ΔG) of folding we measured (Table 2). When we take into account the non-canonical pairs we revealed in the three-dimensional structures, a trend appears. The two pri-miRNAs that form non-canonical pairs and have G-C or C-G as terminal stem pairs (pri-mir-300 and pri-mir-208a) are the most stable, whereas the ones that do not form non-canonical base pairs and contain A-U or U-A canonical stem pairs (pri-mir-320b-2 and pri-mir-19b-2) are the least stable (FIG. 4b). Most other pri-miRNA sequences that either contain non-canonical pairs but A-U/U-A stem pairs (pri-mir-340 and pri-mir-449c), or form no non-canonical pairs but with G-C/C-G stem pairs (pri-mir-202) are intermediate in stability. The pri-mir-378a apical junction/loop contains a C-A non-canonical pair that is defined by a single hydrogen bond and thereby displays a AG similar to those from the least stable group. Together, these data suggest that the non-canonical pairs at pri-miRNA apical junctions contribute to their structural stability in solution.

Human Pri-miRNAs Favor U-U and G-A Pairs at their Apical Junctions

We next estimated the abundance of non-canonical pairs at pri-miRNA apical junctions by analyzing all human pri-miRNA loop sequences. Among 1,881 such sequences, 340 contain U residues at both the 5′ and 3′ ends that are most likely to pair like in the pri-miR-340, pri-miR-300, and pri-miR-449c structures (FIG. 4c). The U-U pair is the most abundant among all possible combinations at these positions, whereas the expected occurrence by chance is 181. This enrichment is highly significant, as the probability of observing U-U 340 times by chance is 3×10⁻²⁸ times lower than that for 181 times. The second most abundant combination is 5′-G and 3′-A, observed 245 times, 1×10⁻¹⁶ fold less likely to occur by chance than the odd for most probable count of 139. The loop sequence counts of other terminal combinations such as C-A (observed 122 times) are less substantially different from that expected by chance (109 times, P₁₂₂-P₁₀₉=0.42). Therefore, we conclude that human pri-miRNAs favor U-U and G-A pairs immediately next to the hairpin stem.

Intriguingly, U-U and G-A are known to stabilize hairpin loops when serving as the closing pairs (16). Our pri-miRNA loop library was constructed partially based on secondary structure predictions that have taken into consideration the stabilizing effects of U-U and G-A pairs. We do not think this small bonus energy term is responsible for the enrichment of U-U and G-A as closing pairs in pri-miRNA apical loops, as for most pri-miRNAs the loop sequences are defined by strong canonical base pairs as part of the pri-miRNA hairpin stem. Additionally, other non-canonical pairs, such as G-G, C-A and A-C, are also known to be stabilizing (although to slightly less extents), but they are not enriched in pri-miRNA apical junctions. This result suggests that U-U and G-A non-canonical pairs are favored by pri-miRNA apical junctions, possibly for their stabilizing effects and/or specific geometric features.

Pri-miRNA Loops Share Structural Features with Other RNAs

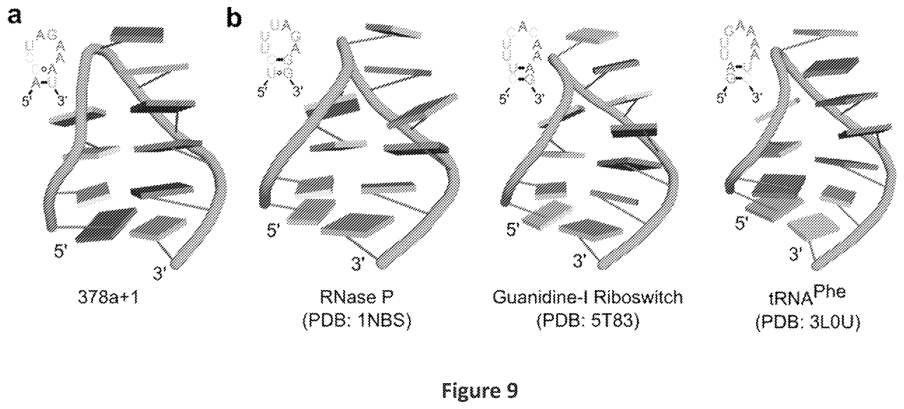

We asked whether the loop conformations we uncovered were unique to pri-miRNAs or shared with other RNA stem-loops. To address this question, we threaded RNA hairpin sequences from the PDB onto our pri-miRNA structures and then calculated the RMSD between the threaded pose and the original PDB conformation (see Methods section). For pri-miR-378a we identified three loops that are slightly shorter (6- or 7-nt) and differ in sequence but retain a highly similar fold (FIG. 9). Comparing these structures reveals a generalized loop motif, which we call 3′-purine-rich stack (FIG. 9b). In a 3′-purine-rich stack, 4-5 mostly purine bases on the 3′ side of the loop stack with each other, on top of the helical stem. One or two pyrimidines may be found in positions furthest from the stem. On the 5′ side of the loop, two or three pyrimidine residues, most often uridines, serve as linkers between the stacked residues and the stem. These linker pyrimidines form hydrogen bonds with stacked purines, sometimes non-canonical base pairs, which further stabilize the whole loop. More broadly, in the pri-miR-320b-2 structure the three purines in the UGAA tetraloop stack with each other and on top of the neighboring U-A pair, essentially forming a 3′ purine stack. Many pri-miRNA and other hairpin loops contain sequences consistent with a 3′ purine stack. Overall, these observations suggest that the pri-miRNA loop structures are not necessarily unique to pri-miRNAs, also consistent with the previous reports that DGCR8 and Drosha interact with many other cellular RNAs (17-21).

### Asymmetric Conformational Flexibility of Pri-miRNA Apical Loops

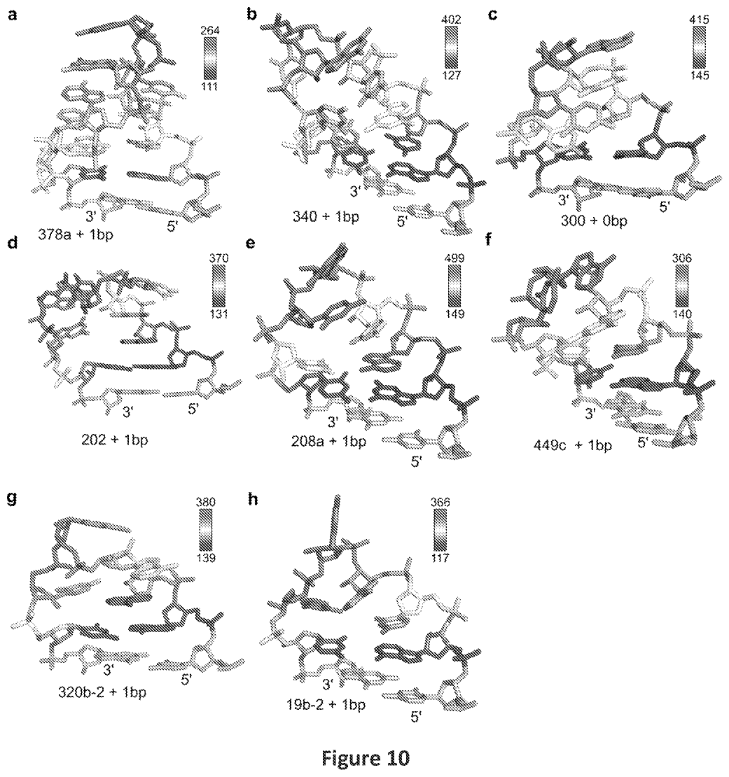

Structural stability and dynamics are likely to be important for pri-miRNA junctions and loops for at least two reasons. First, common conformational features are expected to be stable. Second, dynamic regions make it easier to avoid steric hindrance when binding processing proteins and to adopt conformations favorable for processing. To investigate this, we first reviewed the atomic displacement parameters (ADPs, also known as the temperature or B-factors) refined during structure determination. Not surprisingly, residues at the top of the loop have large ADPs, suggesting that they are highly dynamic; whereas residues close to the stems, which are involved in common structural features such as non-canonical pairs and base stacking, tend to have lower ADPs (FIG. 10). Importantly, most loops display a trend toward higher stability at the 5′ region of the loop and more flexibility in the 3′ region, with exception of pri-miR-378a. The stacked 5′ residues are consistently more stable than the 3′ nucleotides. To further compare ADPs between structures, we calculated the average ADP per residue and then plotted them on the same scale (FIG. 4d). The peak in ADPs is consistently located near the middle to 3′ end of the loop across most structures. It is interesting to note that the UGU motif previous identified to be important for efficient processing (5, 10) is located in the 5′ region of the loop.

For a more detailed view into the loop dynamics, we performed molecular dynamics simulation of the pri-miRNA junction and loop nucleotides in explicit solvent. For simplicity, the simulation included only the pri-miRNA residues plus two base pairs from the scaffold, and we restrained the position of the scaffold nucleotides to prevent unwinding of the strand (see Methods for details). We ran the simulations at 300 K for 1 μs and analyzed the resulting trajectories by calculating the root-mean-square fluctuation (RMSF) for each residue (FIG. 4e). These statistics support the trend more obvious that the center to 3′ loop residues sample a wider range of conformations.

### Correlation Between Rhed-Binding Affinity and Apical Loop Length

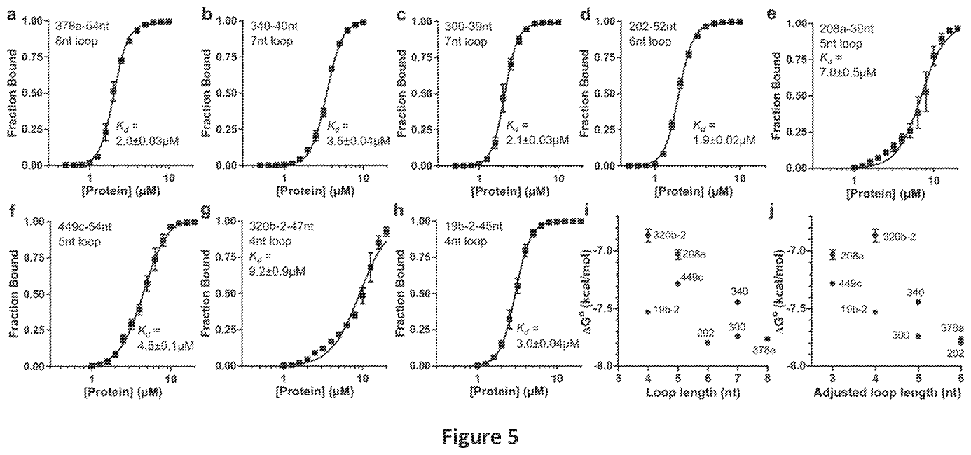

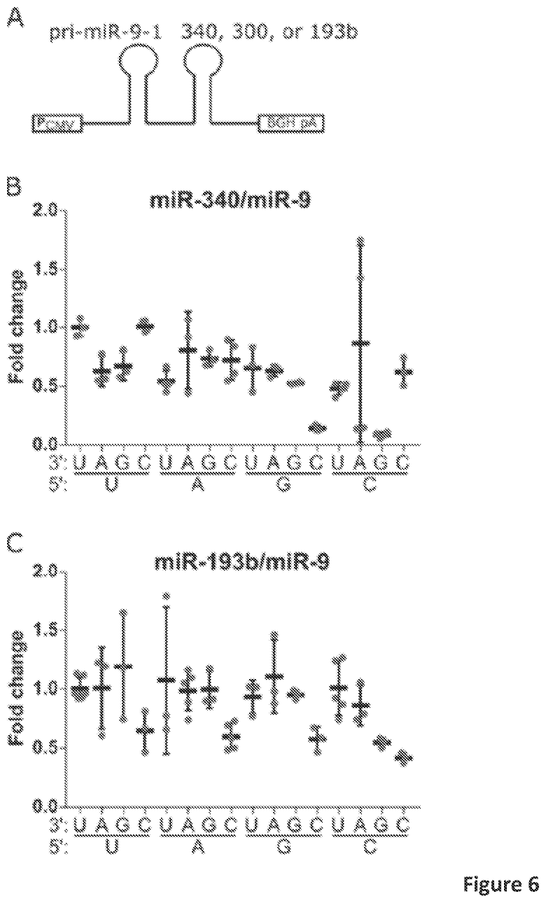

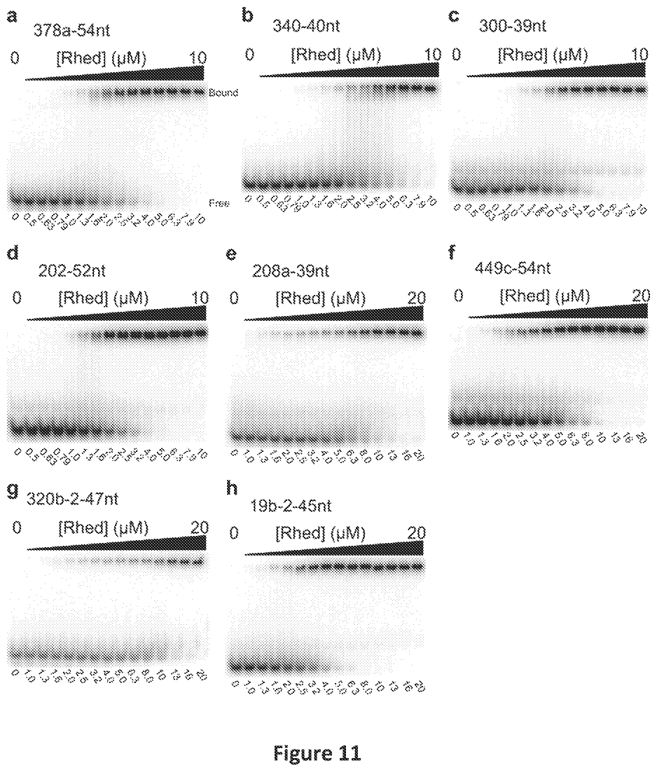

We wondered how the Rhed recognizes all pri-miRNA apical junctions despite differences in loop length. We addressed this question by measuring the affinities of Rhed for pri-miRNA fragments containing the apical loop plus approximately 20 bp from the stem (FIG. 8b-i). We used electrophoresis mobility shift assay (EMSA) to determine the Rhed dissociation constant (K_{d}) for each RNA (FIG. 11). The Rhed bound all pri-miRNA fragments with K_{d} ranging from 1.9 to 9.2 μM (FIG. 5a-h). Such differences could be important for the recognition, especially when pri-miRNAs compete for the processing machinery. We plotted the AG of binding versus the overall loop length (FIG. 5i) and noticed a trend toward tighter binding of the longer loops. This trend became more obvious when we corrected the loop length based on our 3D structures (length minus number of residues involved in non-canonical pairs, FIG. 5j). Our results provide a biochemical explanation for pri-miRNA loop length preference, although we cannot rule out the possibility that differences in the pri-miRNA stems also contribute to the range of Rhed affinities. We note that pri-miR-340, which contains the UGU motif at the 5′ side of the loop, binds the Rhed with similar affinity (K_{d}=3.5 μM) to other constructs lacking this sequence.

### Discussion

We provide working embodiments demonstrating a proof-of-concept that scaffold-directed crystallography can be a powerful tool for RNA structural biology. This method is largely analogous to the popular fixed-arm MBP fusion technique, in which a target protein is linked to MBP in a fixed orientation via a continuous alpha-helical linker (22). However, our engineering approach specifically positions the target RNA within a lattice void of the scaffold crystal. Such a design results in several additional advantages: (1) because the target moiety does not disrupt existing lattice contacts, the fusion molecule can be crystalized under the original conditions; (2) since rescreening of a broad array of conditions is unnecessary, a minimal amount of purified fusion RNA is required for crystallization; and (3) the target does not interact with neighboring molecules in the lattice, thereby allowing its structure to closely represent the conformation in solution.

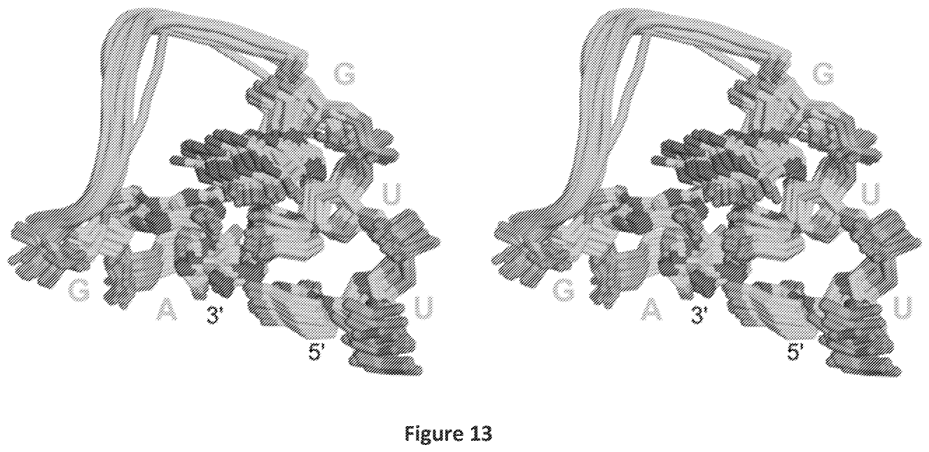

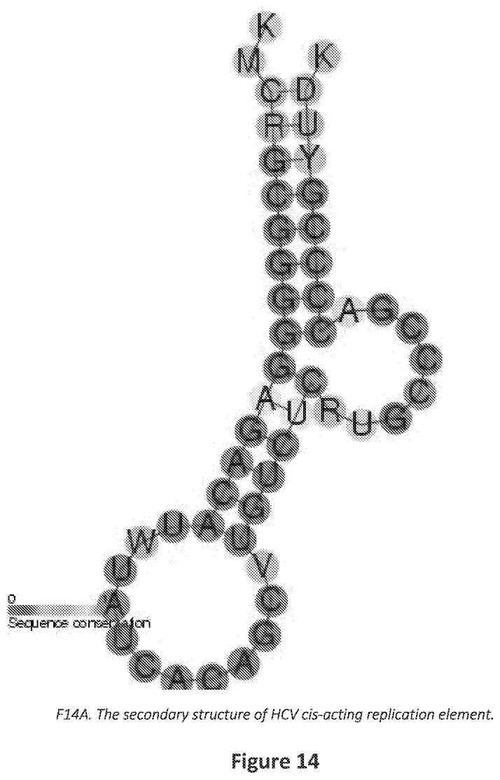

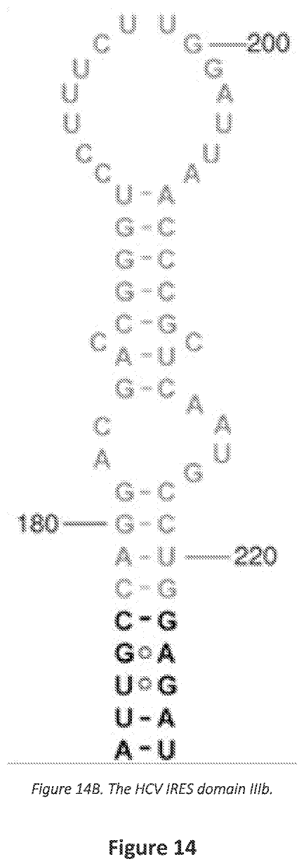

Applying this technique to the problem of pri-miRNA recognition provides an atomic-level survey of eight pri-miRNA apical junction and loop structures. These loops cover the most frequent loop lengths among human pri-miRNAs. These structures collectively reveal a structural consensus that involves a non-canonical base pair closing the apical loop and further base stacking at the 5′ end. This consensus is supported by the previously reported NMR structure for pre-miR-20b (6). The pre-miR-20b stem terminates in a G-U pair and the neighboring 5′ loop nucleotide (G) stacks on top of the pair (FIG. 13). Comparison of the top 20 NMR solutions confirms these are stable features of the molecule. NMR study of pre-miR-21 revealed weak signals corresponding to two tandem U-G/G-U pairs at the apical junction and suggested that the 14-nt apical loop is otherwise unstructured (11). Beyond the apical junctions, the apical loops in our and NMR structures differ in three-dimensional conformation, suggesting that their conformations are not direct specificity determinants. These conformations are relevant to their individual functions. For example, the pri-miR-125a loop can function as an aptamer domain for binding folic acid (23).

The observation of non-canonical pairs at pri-miRNA apical junctions in and of itself has important structural and functional implications. Our optical melting experiments indicate that these pairings contribute to thermodynamic stability of the RNA in solution (FIG. 4b). In particular, U-U and G-A pairs are highly enriched at apical junctions in human pri-miRNAs (FIG. 4c). These pairs are often conserved. For example, the U-U pair in pri-miR-340 is nearly completely conserved, whereas nucleotide variation occurs in all other positions. The only variation of the U-U pair is a substitution by a U-G pair in Pteropus alecto. Therefore, these non-canonical pairs at the apical junction are likely to be important for miRNA maturation, although their exact functions remain to be determined. Microprocessor recognizes a pri-miRNA hairpin by clamping its stem at both ends (24, 25). The optimal pri-miRNA hairpin stem length is estimated to be 35±1 bp, counting in internal non-canonical pairs (10). Our study suggests that terminal non-canonical pairs at apical junctions have to be considered. Previous high-throughput mutagenesis of pri-miR-16-1 indicates that due to the longer-than-optimal stem length and that disruption of canonical pairs at the apical end of the stem increases the Microprocessor cleavage efficiency (10). In pri-miR-16-1, a G-A pair is expected to form and stack at the end of the hairpin stem. In such a scenario, the G-A pair would need to be disrupted along with the neighboring canonical pairs. Such inhibitory effects make possible activation of miRNA maturation by RNA-binding proteins and RNA helicases (26). Conversely, we imagine that in cases where the pri-miRNA helical stem are shorter than optimal, the non-canonical pairs would help the hairpin fits in the Microprocessor complex.

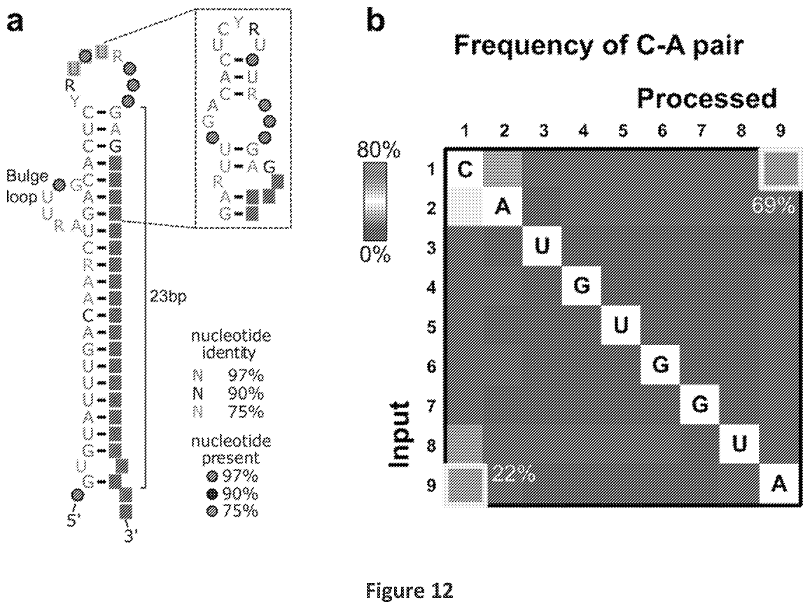

The conformation of the apical junction may also be preferentially recognized by Microprocessor. Indeed, Microprocessor prefers a U-G pair over Watson-Crick base pairs at the 35^{th}-bp position of the pri-miR-30a stem (counting from the basal junction) (10). We re-analyzed another high-throughput mutagenesis data (5) and found that C-A pair is highly enriched at the apical junction among the Microprocessor cleavage products (FIG. 12). Furthermore, the tendency of 5′ loop residues to stack and 3′ loop section to be more flexible allows the UGU motif to be positioned and exposed for recognition by the processing machinery. Further studies are required to test this idea.

Our analysis of human pri-miRNA loop sequences suggests that most of them are shorter than the optimal ≥10-nt. Among the eight pri-miRNAs with loop lengths between 4-8 nt, we observe a correlation between the loop length and free energy change of binding with Rhed (FIG. 5i). The correlation is improved when residues involved in non-canonical pairs are excluded from the calculation of loop length (FIG. 5j). Preferential binding to Rhed would poise a pri-miRNA in an advantageous position for processing, thereby providing a biochemical explanation for the optimal loop length of ≥ 10 nt (7). The differences in ΔG_{binding} to Rhed are within 1 kcal/mol.

We believe such moderate differences can have substantial biological and pathological consequences, especially when Microprocessor becomes limited (in many cancer cells for example). Preferential binding to Microprocessor, as represented by the interaction of apical junctions with Rhed shown here, may generate a hierarchy of processing among pri-miRNAs and helps to determine miRNA expression profiles.

Apical junctions and loops are also part of pre-miRNAs that are exported to the cytoplasm and cleaved by the Dicer ribonuclease in the miRNA maturation pathway. Previous studies have shown that the stem and loop lengths of pre-miRNAs can affect both the Drosha and Dicer cleavage efficiency (8). Further studies are required to understand how the apical junction and loop structures contribute to the Dicer processing step. Furthermore, there is a substantial interest of developing potential therapeutic agents that target pri-miRNA, mRNA and viral RNA hairpin loops (11, 26, 27). Our structures indicate that the pri-miRNA loops contain more structures than expected, which would reduce the entropy penalty for binding. Our crystallization method should allow structure-based design of inhibitors.

### Methods

### Pri-miRNA Apical Loop Analysis

To gauge the approximate size of the apical loops, we downloaded from miRBase (release 21) all annotated human “hairpin” sequences and their genomic coordinates. The miRBase hairpins typically include the pre-miRNA moiety along with a variable number of additional base pairs from the basal stem. For each hairpin, we used the genomic sequence to extend the RNA an equal number of nucleotides at the 5′ and 3′ ends until the total length equaled 150 nt. This 150-nt window contained the full pri-miRNA hairpin, plus some single-stranded RNA on either side of the basal junction. We then generated predicted secondary structures for all pri-miRNA hairpins using MFOLD (14), and generally retained the top scoring structures (i.e. with the lowest predicted free energy of folding). We manually reviewed all the predictions to ensure they reflected the expected hairpin structure with mature miRNA sequences derived from either or both strands of the stem: in cases where mfold predicted alternative conformations, we selected the structure with the lowest free energy that contained a stem length of approximately three helical turns. We manually compared the secondary structures with those from the miRBase and also eliminated 1-2 base pairs in the hairpin that are isolated from the stem and thereby deemed to be unstable.

### PDB Mining and Identification of YdaO Crystallization Scaffold

We first filtered the PDB to obtain X-ray structures containing only RNA molecules (no protein or DNA). To identify voids in the crystal lattices, we wrote a PyMOL script that implemented a grid search algorithm in the following steps. (1) Generate a 3×3×3 block of unit cells (i.e. 27 copies of the unit cell). The unit cell at the center of this block sees all possible lattice voids, either internally or between unit cells. (2) Using three unit vectors along each of the unit cell axis (i.e. a, b, and c vectors of length 1 Å), iteratively generate grid points of the form 5*i*a+5*j*b+5*k*c for integer values of i, j, k less than the respective unit cell edge length divided by 5. This gives grid points with 5 Å spacing. (3) For each grid point, calculate the distances to all C1′ atoms in the super cell and identify the shortest as R_{local}. For each structure, identify the grid point with the largest R_{local} as Rₘₐₓ.

To find suitable scaffolds, we then manually reviewed the structures with large Rₘₐₓ values and a single molecule in the asymmetric unit. We traced the chain looking for any stem-loop that projected into the cavity in the lattice. Amongst several hundred candidates reviewed, only the P2 stem-loop from the YdaO riboswitch (PDB ID: 4QK8) met these conditions (15).

### Preparation of YdaO WT and Pri-miR-9-1 Fusion RNA and Native Gel Electrophoresis

We initially designed the W.T. YdaO construct to contain a T7 promoter sequence at the 5′ end and HDV ribozyme on the 3′ side, along with flanking EcoRI and BamHI restriction sites. This fragment was synthesized as a gene block (IDT), double digested and cloned into the pUC19 plasmid. The clone was verified by Sanger sequencing. To replace the P2 loop nucleotides with the pri-miRNA stem-loop, we used a two-round PCR protocol. All reactions were performed with Q5 high-fidelity DNA polymerase (New England Biolabs) following the manufacture's recommended reaction setup and cycling conditions. All reactions contained the same reverse primer, which annealed to the 3′ end of HDV and contained the BamHI site (5′-CGTGGATCCGGTCCCATTC-3′) (SEQ ID NO: 2). For the first PCR, the forward primer contained the pri-miRNA sequence plus around 20 nt upstream and downstream on the scaffold. The forward primers for pri-miR-9-1 fusions were

(SEQ ID NO: 3)
5′-CTATAGGTTGCCGAATCCGTGGTGTGGAGTCTGGTACGGAGGAACC
GCTTTTTG-3′ (pri-miR-9-1 + 0 bp);
(SEQ ID NO: 4)
5′-CTATAGGTTGCCGAATCCAGTGGTGTGGAGTCTTGGTACGGAGGAA
CCGCTTTTTG-3′ (pri-miR-9-1 + 1 bp);
(SEQ ID NO: 5)
5′-CTATAGGTTGCCGAATCCGAGTGGTGTGGAGTCTTCGGTACGGAGG
AACCGCTTTTTG-3′ (pri-miR-9-1 + 2 bp);
(SEQ ID NO: 6)
5′-CTATAGGTTGCCGAATCCAGAGTGGTGTGGAGTCTTCTGGTACGGA
GGAACCGCTTTTTG-3′ (pri-miR-9-1 + 3 bp).

This PCR product was gel-purified and 1 μL was used as template for the second-round PCR. All reactions contained the same reverse primer and a forward primer (5′-GCAGAATTCTAATACGACTCACTATAGGTTGCCGAATCC-3′) (SEQ ID NO: 7), which annealed to the common scaffold residues (bold) and added the T7-promoter (italic) and EcoRI site (underlined). The second-round PCR product was gel-purified, digested with EcoRI and BamHI, and ligated into pUC19. Clones containing the desired insert were sequence-verified.

For WT YdaO and pri-miR-9-1 fusion constructs we prepared maxiprep plasmids and linearized them by overnight digestion with BamHI. Transcription reactions contained ˜400 μg linearized template, 40 mM Tris pH7.5, 25 mM MgCl₂, 4 mM DTT, 2 mM spermidine, 40 μg inorganic pyrophosphatase (Sigma), 0.7 mg T7 RNA polymerase, and 3 mM each NTP in a total volume of 5 mL. After 4.5 hr of incubation at 37° C., the final MgCl₂ concentration was adjusted to 40 mM, and the reactions were incubated for additional 45 min. Despite the elevated Mg²⁺ concentration, we observed only partial cleavage by the HDV ribozyme. Reactions were ethanol precipitated and purified over denaturing 10% polyacrylamide slab gels. The desired product was visualized by UV shadowing and excised from the gel. Gel pieces were crushed and extracted overnight in 30 mL TEN buffer (150 mM NaCl, 20 mM Tris pH 7.5, 1 mM EDTA) at 4° C. We then spun down the gel pieces and concentrated the RNA in an Amicon Ultra-15 centrifugal filter unit with 10-kDa molecular weight cutoff (MWCO). RNA was buffer-exchanged three times into 10 mM HEPES pH7.5 and concentrated to ˜50 μL final volume.

For analysis on a native gel, 5 μM RNA stock solutions were prepared by dilution of the purified RNA into 5 mM Tris pH 7.0. Next, 2.5 μL RNA was mixed with an equal volume of 2× annealing buffer containing 35 mM Tris pH 7.0, 100 mM KCl, 10 mM MgCl₂, and 20 μM c-di-AMP (Sigma). The mixtures were heated at 90° C. for 1 min followed by snap cooling on ice and then a 15-min incubation at 37° C. The annealed RNA was mixed with a 2× loading dye containing 40 mM Tris pH 7.0, 50 mM KCl, 5 mM MgCl₂, 20% (v/v) glycerol, and xylene cyanol, and analyzed on a 10% polyacrylamide gel with Tris-borate (TB) running buffer. The gel was stained in Sybr Green 11 and scanned on a Typhoon 9410 Variable Mode Imager (GE Healthcare).

### Preparation of Pri-miRNA-YdaO Fusions for Crystallization

Given the poor HDV self-cleavage efficiency we observed for the pri-miR-9-1 fusions, we elected to change strategy. Instead of employing a ribozyme to create homogeneous 3′ ends, we used PCR to generate transcription templates in which the two 5′ residues on the anti-sense DNA strand were 2′-O-methylated. The modifications have been shown to reduce un-templated nucleotide addition by T7 RNA polymerase (28). We utilized a three-round PCR approach to create the transcription templates. All reactions below contained the same reverse primer, 5′-mCmUCCTTCCTTTATTGCCTCC-3′ (SEQ ID NO: 8), where ‘m’ indicates 2′-O-methylation. For the first round of PCR, we set up a 50 μL reaction with Q5 polymerase to amplify the 3′ fragment of YdaO with the forward primer 5′-GGTACGGAGGAACCGCTTTTTG-3′ (SEQ ID NO: 9) and performed 30 cycles of amplification. The product was gel purified and 1 μL was used as template for the next round. In the second-round PCR, we used a unique forward primer for each construct containing the pri-miRNA loop and stem sequence which annealed to the 3′ YdaO fragment from the first stage. The primer sequences were

(SEQ ID NO: 10)
5′-CTATAGGTTGCCGAATCCATATGTGGTACGGAGGAACCGCTTTTT
G-3′ (19b-2 + 1 bp);
(SEQ ID NO: 11)
5′-CTATAGGTTGCCGAATCCGATCTGGCGGTACGGAGGAACCGCTTTT
TG-3′ (202 + 1 bp);
(SEQ ID NO: 12)
5′-CTATAGGTTGCCGAATCCGATGCTCGGTACGGAGGAACCGCTTTTT
G-3′ (208a + 1 bp);
(SEQ ID NO: 13)
5′-CTATAGGTTGCCGAATCCCTTTACTTGGGTACGGAGGAACCGCTTT
TTG-3′ (300 + 1 bp);
(SEQ ID NO: 14)
5′-CTATAGGTTGCCGAATCCAAAGTTGGTACGGAGGAACCGCTTTTT
G-3′ (320b-2 + 1 bp);
(SEQ ID NO: 15)
5′-CTATAGGTTGCCGAATCCATGTCGTTTGGTACGGAGGAACCGCTTT
TTG-3′ (340 + 1 bp);
(SEQ ID NO: 16)
5′-CTATAGGTTGCCGAATCCACCTAGAAATGGTACGGAGGAACCGCTT
TTTG-3′ (378a + 1 bp);
and
(SEQ ID NO: 17)
5′-CTATAGGTTGCCGAATCCATGATTTGGTACGGAGGAACCGCTTTTT
G-3′ (449c + 1 bp).

This reaction was also 50 μL and used Q5 polymerase for 30 cycles. The product from the second-round PCR was analyzed by agarose gel electrophoresis to confirm amplification, and 40 μL of the reaction was used as template for the third-round PCR without further purification. The 2-mL PCR reactions used the Phusion high-fidelity DNA polymerase (Thermo-Fisher) and the forward primer 5′-GCAGAATTCTAATACGACTCACTATAGGTTGCCGAATCC-3′, (SEQ ID NO: 18) and was run for 35 cycles.

The third-stage PCR product was purified over a HiTrap Q HP column (GE Healthcare). Buffer A contained 10 mM NaCl and 10 mM HEPES pH 7.5; Buffer B was identical but with 2 M NaCl. The column was equilibrated with 20% Buffer B and the desired DNA product was eluted with a linear gradient to 50% B over 10 min at 2 ml/min. We analyzed the peak fractions on an agarose gel to confirm they contained a single band of the correct size. The peak fractions were then pooled and concentrated in an Amicon filter unit (10 kDa MWCO), and then washed with water to remove excess salt. The concentration of the DNA template (˜200 μL final volume) was determined by UV absorbance.

Transcription reactions were set up as described above for pri-miR-9-1 fusions, but in a 10-mL volume and containing 2.8 fmol DNA template. Reactions were run for 4 hr at 37° C. followed by phenol-chloroform extraction. The transcription was concentrated in an Amicon filter unit (10 kDa MWCO) and washed with 0.1 M trimethylamine-acetic acid (TEAA) pH 7.0. The RNA (˜2 mL) was injected onto a Waters XTerra MS C18 reverse phase HPLC column (3.5 μm particle size, 4.6×150 mm in dimension) thermostated at 54° C. TEAA and 100% acetonitrile were used as mobile phases. The column was washed with 6% acetonitrile and the RNA eluted with a gradient to 17% acetonitrile over 80 min at 0.4 ml/min. Peak fractions were analyzed on denaturing 10% polyacrylamide gels. Pure fractions were pooled and buffer-exchanged into 10 mM HEPES pH 7.0 using an Amicon filter unit. The RNA was concentrated to <50 μL final volume and the concentration determined by UV absorbance.

### Crystallization, Data Collection, and Structure Determination

All RNA-c-diAMP complexes were prepared as described (15). Briefly, a solution containing 0.5 mM RNA, 1 mM c-di-AMP, 100 mM KCl, 10 mM MgCl₂, and 20 mM HEPES pH 7.0 was heated to 90° C. for 1 min, snap cooled on ice, and equilibrated for 15 min at 37° C. immediately prior to crystallization. Screening was performed in 24-well plates containing 0.5 mL well solution; the hanging drops consisted of 1 μL RNA plus 1 μL well solution. Plates were incubated at room temperature, and crystals generally grew to full size (100 μm to over 200 μm) within one week. For 19b-2+1 bp, the well solution contained 1.7 M (NH₄)₂SO₄, 0.2 M Li₂SO₄, and 0.1 M HEPES pH 7.1. For 202+1 bp, 208a+1 bp, and 320b-2+1 bp, the well contained 1.9 M (NH₄)₂SO₄, 0.2 M Li₂SO₄, and 0.1 M HEPES pH 7.4. The well solution for 378a+0 bp contained 1.7 M (NH₄)₂SO₄, 0.2 M Li₂SO₄, and 0.1 M HEPES pH 7.4. For the remaining constructs crystallization was performed in 96-well plates with hanging drops consisting of 0.4 μL RNA plus 0.4 μL well solution. For 300+1 bp, the well solution contained 1.88 M (NH₄)₂SO₄, 0.248 M Li₂SO₄, and 0.1 M HEPES pH 7.4, and for 300+0 bp it held 1.90 M (NH₄)₂SO₄, 0.158 M Li₂SO₄, and 0.1 M HEPES pH 7.4 Construct 340+1 bp crystallized from a well solution containing 1.89 M (NH₄)₂SO₄, 0.214 M Li₂SO₄, and 0.1 M HEPES pH 7.4. Construct 378a+1 bp crystallized from 1.63 M (NH₄)₂SO₄, 0.272 M Li₂SO₄, and 0.1 M HEPES pH 7.4. For construct 449c+1 bp, the well contained 1.89 M (NH₄)₂SO₄, 0.128 M Li₂SO₄, and 0.1 M HEPES pH 7.4

All crystals were briefly soaked in a cryoprotectant solution containing 20% (w/v) PEG 3350, 20% (v/v) glycerol, 0.2 M (NH₄)₂SO₄, 0.2 M Li₂SO₄, and 0.1 M HEPES pH 7.3, and then flash-frozen in liquid nitrogen. Data were collected at 100 K at the Advanced Photon Source Beamline 24-ID-C or the Advanced Light Source Beamline 8.3.1. For all constructs we collected a native dataset at a wavelength of ˜1 Å. For 320b-2+1 bp, 378a+0 bp, and 449c+1 bp, we measured phosphorous anomalous scattering by collecting additional high-redundancy datasets at 1.9 Å from 1, 2, or 3 crystals, respectively. Data were indexed, integrated, and scaled using XDS (29).

Where anomalous data was available, we generated partially-experimental phases using a combined molecular-replacement/single anomalous dispersion approach (MR-SAD). The molecular replacement model consisted of the YdaO c-di-AMP riboswitch structure (PDB ID: 4QK8) with the GAAA tetraloop on the P2 stem removed from the model. Phases were obtained using the default settings in the Phaser-MR protocol in Phenix (30).

For all constructs we obtained an initial solution by performing a rigid body fit of the MR model (above) to data using Phenix (including experimental phase restraints where available). This produced an excellent initial model with R_{work}<30%. We then inspected the electron density map in region of the P2 stem. For all RNAs, additional density for the missing base-pair and loop could clearly be seen in the 2Fₒ-F_{c} and difference maps. We then modeled in the missing residues in Coot (31). In cases where the density was unclear, we stopped modeling with an incomplete loop and performed an additional round of coordinate, ADP, and TLS parameter refinement with Phenix. This typically revealed additional density for the missing residues. Once the loop was completely modeled, we performed subsequent rounds of refinement and manual adjustment as above until reasonable R factors and model geometry were obtained.

Simulated annealing composite omit maps were calculated in Phenix (FIG. 7). In the case of 19b-2+1 bp, 202+1 bp, 320b-2+1 bp, 340+1 bp, and 378a+1 bp, the standard annealing temperature (5000° C.) and other default parameters produced reasonable maps. However, for 300+0 bp, 300+1 bp and 378a+0 bp the default settings generated noisy maps with regions of broken density. To improve the quality of the maps, we reduced the annealing temperature to 1000° C. and excluded the bulk solvent mask from the omitted regions. This type of composite omit map is known as Polder map and prevents the solvent mask from obscuring weaker density (32).

### Comparison to Known RNA Loop Structures in the PDB

To identify RNA loops in the PDB with structural similarity to our pri-miRNA loop models, we first extracted the coordinates for the pri-miRNA apical junctions and loops. The search pool was the same set of RNA structures used to identify crystallization scaffolds above. For each structure from the PDB set, we used DSSR to identify all hairpin loops. We extracted the RNA sequence from each hairpin loop, and eliminated loops shorter than the pri-miRNA sequence. For loops longer than the pri-miRNA, we used a sliding window to obtain all fragments of the loop with the same length. Each loop sequence was then threaded onto the pri-miRNA model using the “ma_thread” routine in Rosetta (33). Using a PyMOL script, we aligned the resulting threaded model to the original hairpin loop and calculated the RMSD between the two models. We aggregated and sorted the RMSD data from all PDB structures and manually inspected loops with small RMSD to find hits with structural similarity.

### Optical Melting

RNA for optical melting experiments were transcribed in vitro from synthetic DNA templates (IDT). The oligonucleotide template sequences used were 5′-GGAACACATATGTTCCTATAGTGAGTCGTATTA-3′ (19b-2) (SEQ ID NO: 19), 5′-GGAACGCCAGATCGTTCCTATAGTGAGTCGTATTA-3′ (202) (SEQ ID NO: 20), 5′-GGAACGAGCATCGTTCCTATAGTGAGTCGTATTA-3′ (208a) (SEQ ID NO: 21), 5′-GGAACCAAGTAAAGGTTCCTATAGTGAGTCGTATTA-3′ (300) (SEQ ID NO: 22), 5′-GGAACAACTTTGTTCCTATAGTGAGTCGTATTA-3′ (320b-2) (SEQ ID NO: 23), 5′-GGAACAAACGACATGTTCCTATAGTGAGTCGTATTA-3′ (340) (SEQ ID NO: 24), 5′-GGAACATTTCTAGGTGTTCCTATAGTGAGTCGTATTA-3′ (378a) (SEQ ID NO: 25), and 5′-GGAACAAATCATGTTCCTATAGTGAGTCGTATTA-3′ (449c) (SEQ ID NO: 26), with the T7 promoter shown in italics and the pri-miRNA junction/loop segment in bold. Templates were annealed with a second strand complementary to T7 promoter and added to large-scale (10 mL) transcription reactions as described above. Reactions were ethanol precipitated, purified over 20% polyacrylamide denaturing gels. The desired band recovered by UV shadowing. Following gel extraction, samples were buffer exchanged into water and concentrated in an Amicon centrifugal filter device.

For each RNA, a set of 6 dilutions were prepared in 50 mM NaCl and 10 mM sodium cacodylate pH 7.0, such that the initial absorbance ranged from ˜1.0 to 0.1 AU. The samples were annealed by heating to 95° C. for 1 min and snap cooling on ice, followed by equilibration to 12° C. Melting measurements were performed with a Cary Bio300 UV-visible spectrophotometer equipped with a Peltier-type temperature controlled sample changer. The absorbance at 260 nm was recorded while the RNA was heated from 12° C. to 92° C. at a rate of 0.8° C./min. Melting curves were analyzed using Prism (GraphPad, version 7) and fit with the equation

$$A = \frac{{e}^{( \frac{Δ S}{R} - \frac{Δ H}{RT} )}}{1 + {e}^{( \frac{Δ S}{R} - \frac{Δ H}{RT} )}} ( {m}_{f} T + {b}_{f} ) + \frac{1}{1 + {e}^{( \frac{Δ S}{R} - \frac{Δ H}{RT} )}} ( {m}_{u} T + {b}_{u} ) ,$$

where the absorbance (A) is approximated as a function of temperature (T). The changes in entropy (ΔS) and enthalpy (ΔH) were fit as well as the slope (m) and y-intercept (b) for both the double-stranded (m_{f} and b_{f}) and single-stranded (mᵤ and bᵤ) linear regions. The melting temperatures and thermodynamic parameters at 37° C. were then derived from these parameters (Table 2).

### Electrophoresis Mobility Shift Assay

Human heme-bound Rhed protein was over-expressed in E. coli and purified using ion exchange and size exclusion chromatography, as previously described (25). Radiolabeled pri-miRNA stem-loops (FIG. 8b-i) were prepared by in vitro transcription. DNA templates consisted of anti-sense oligonucleotides covering the desired sequence plus the T7 promoter, annealed to a sense oligo with the T7 promoter sequence (34). Each 20-μL transcription reaction contained 50 fmol template, 40 mM Tris pH 7.5, 25 mM MgCl₂, 4 mM DTT, 2 mM spermidine, 2 μg T7 RNA polymerase, 0.5 mM ATP, 3 mM each of UTP, CTP, and GTP, and 3 nmol α-³²P-ATP (10 μCi). Transcriptions were run at 37° C. for 2 hr and the RNA purified over a denaturing 15% polyacrylamide gel. The RNA were extracted overnight at 4° C. in TEN buffer, isopropanol-precipitated, and resuspended in 40 μL water.

We adopted a recently reported EMSA procedure to examine Rhed-pri-miRNA interactions (35). The RNAs were diluted in 100 mM NaCl, 20 mM Tris pH 8.0 and heated at 90° C. for 1 min followed by snap cooling on ice. The annealed RNA was added to binding reactions containing 10% (v/v) glycerol, 0.1 mg/ml yeast tRNA, 0.1 mg/ml BSA, 5 μg/ml heparin, 0.01% (v/v) octylphenoxypolyethoxyethanol (IGEPAL CA-630), 0.25 unit RNase-OUT ribonuclease inhibitor, xylene cyanol, 20 mM Tris pH 8.0, and 0-20 μM Rhed protein. The final salt concentration of the solution was 150 mM NaCl. Binding reactions were incubated at room temperature for 30 min prior to loading on a 10% polyacrylamide gel. Both the gel and the running buffer contained 80 mM NaCl, 89.2 mM Tris base, and 89.0 mM boric acid (pH 8.2 final). Gels were run at 110 V for 45 min at 4° C., and then dried and exposed to a storage phosphor screen. Screens were subsequently scanned on a Typhoon scanner (GE Healthcare). The free and bound RNA bands were quantified using Quantity One software (BioRad) and fit with the Hill equation in Prism.

### Molecular Dynamics Simulations

Coordinates corresponding to the pri-miRNA residues plus two G-C pairs from the P2 stem of the scaffold were extracted from each crystal structure. Hydrogens were added to the model in GROMACS (36), and the RNA was dissolved in a truncated dodecahedral box with TIP3P water molecules. The box was sufficiently large to space the RNA at least 1 nm from any periodic copy of itself. Next, K⁺ and Cl⁻ ions were added to the system to neutralize the net charge and give a final KCl concentration of 0.1 M. The CHARMM27 force field, Verlet cutoff scheme, and particle-mesh Ewald electrostatics were employed for all calculations. The system was energy minimized until the maximum force acting on any atom was less than 900 kJ/mol/nm. The final potential energy of the system was in the range of −1.3×10⁵ kJ/mol.

Next the system was initially equilibrated in two steps, first in the NVT ensemble and then in the NPT ensemble. Both equilibrium simulations were ran at 300 K over 2 ns using a 2-fs time step. During NVT, temperature was controlled by velocity rescaling. For NPT, the Parnnello-Rahman barostat was used to maintain pressure at 1 bar. For production MD runs, position restraints were applied to the G-C pairs from the scaffold, and all pri-miRNA nucleotides were unrestrained. All production simulations were run in NPT with 2-fs time steps for a total of 1 μs. Trajectories were analyzed using the rmsf and clustering functions in GROMACS.

### Reanalysis of Pri-miR-223 High-Throughput Processing Assays

Sequencing data from the previously reported processing assay for pri-miRNA-223 were downloaded from the Sequence Read Archive (accession number: SRA051323) (5). Reads corresponding to pri-miR-223 were aligned using Bowtie2 (37). Any reads containing unknown nucleotides were eliminated. Reads from the input or selection libraries were separated by their corresponding barcode and counted with Python.

TABLE 1
Data collection and refinement statistics for pri-miRNA loop fusion
structures.

| | | | | | | | | | |
|---|---|---|---|---|---|---|---|---|---|
| | | 378a + | 340 + | 300 + | | 208a + | | | 19b-2 + |
| RNA | 378a + 0 bp | 1 bp | 1 bp | 0 bp | 202 + 1 bp | 1 bp | 449c + 1 bp | 320b-2 + 1 bp | 1 bp |

Data Collection

| | | | | | | | | | | | | | |
|---|---|---|---|---|---|---|---|---|---|---|---|---|---|
| Data set | Native | SAD | Native | Native | Native | Native | SAD | Native | SAD | Native | Native | SAD | Native |
| No. crystals | 1 | 1 | 1 | 1 | 1 | 2 | 1 | 1 | 2 | 1 | 1 | 3 | 1 |
| Wavelength (Å) | 1.116 | 1.907 | 1.116 | 1.116 | 1.116 | 0.9202 | 1.907 | 0.9791 | 1.907 | 0.9791 | 0.9202 | 1.907 | 0.9791 |
| Data range (°) | 360 | 3,240 | 360 | 720 | 360 | 1,080 | 4,320 | 180 | 4,320 | 360 | 720 | 4,920 | 360 |
| Space group | P3₁21 | P3₁21 | P3₁21 | P3₁21 | P3₁21 | P3₁21 | P3₁21 | P3₁21 | P3₁21 | P3₁21 | P3₁21 | P3₁21 | P3₁21 |
| a, b, c (Å) | 113.8, | 114.0, | 114.9, | 114.8, | 113.1, | 114.9, | 115.1, | 114.6, | 115.0, | 114.7, | 114.6, | 114.9, | 115.3, |
| | 113.8, | 114.0, 115.1 | 114.9, | 114.8, | 113.1, | 114.9, | 115.1, | 114.6, | 115.0, | 114.7, | 114.6, | 114.9, | 115.3, |
| | 115.0 | | 114.7 | 115.6 | 114.1 | 115.3 | 115.3 | 115.1 | 115.1 | 114.8 | 115.3 | 115.6 | 115.3 |
| α, β, γ (°) | 90, 90, | 90, 90, 120 | 90, 90, | 90, 90, | 90, 90, | 90, 90, | 90, 90, | 90, 90, | 90, 90, | 90, 90, | 90, 90, | 90, 90, | 90, 90, |
| | 120 | | 120 | 120 | 120 | 120 | 120 | 120 | 120 | 120 | 120 | 120 | 120 |
| Resolution (Å) | 74.8 - | 74.9-3.18 | 75.2 - | 75.4- | 74.3- | 99.6- | 75.4- | 75.2 - | 75.3 - | 75.1 - | 75.2 - | 99.5- | 75.5- |
| | 2.79 | (3.29-3.18) | 2.95 | 2.99 | 3.08 | 2.71 | 2.95 | 2.80 | 3.59 | 2.95 | 3.12 | 3.17 | 2.85 |
| | (2.89- | | (3.05- | (3.10 - | (3.19- | (2.81 - | (3.05- | (2.90- | (3.71 - | (3.06- | (3.23 - | (3.28- | (2.92 - |
| | 2.79) | | 2.95) | 2.99) | 3.08) | 2.71) | 2.95) | 2.80) | 3.59) | 2.95) | 3.12) | 3.17) | 2.85) |
| Rₘₑₐₛ (%)¹ | 5.9 (140) | 16.7 (195) | 6.7 | 10.1 | 9.2 (143) | 10.6 | 9.6 (146) | 6.2 (158) | 10.6 | 9.0 (220) | 12.2 | 16.0 | 6.1 |
| | | | (154) | (202) | | (217) | | | (189) | | (169) | (170) | (162) |
| R_{p.i.m.} (%)¹ | 1.4 | 1.4 (16.0) | 1.5 | 1.6 (32.4) | 2.1 | 1.4 | 0.7 | 2.0 | 0.8 | 2.0 | 2.8 | 1.1 | 1.4 |
| | (32.7) | | (34.6) | | (34.4) | (33.6) | (17.0) | (49.9) | (22.2) | (47.6) | (37.9) | (20.9) | (35.8) |
| I/σ | 27.7 | 31.4 (3.9) | 25.5 | 27.1 (2.4) | 18.9 | 29.9 | 64.2 | 21.1 | 44.8 | 23.0 | 14.9 | 42.1 | 32.8 |
| | (2.1) | | (1.9) | | (1.7) | (2.1) | (3.5) | (1.5) | (2.5) | (2.0) | (1.7) | (2.9) | (2.02) |
| CC_{1/2} | 100 | 100 (87.6) | 100 | 100 | 100 | 99.6 | 100 | 99.8 | 100 | 100 | 99.8 | 99.9 | 99.9 |
| | (84.8) | | (84.0) | (84.8) | (90.3) | (81.5) | (93.3) | (76.6) | (95.0) | (83.2) | (83.2) | (92.4) | (81.6) |
| Completeness | 99.1 | 99.9 (99.1) | 99.8 | 99.9 | 99.2 | 99.9 | 99.9 | 99.9 | 99.5 | 99.9 | 99.6 | 99.7 | 100.0 |
| (%) | (88.5) | | (97.5) | (100) | (92.4) | (99.0) | (99.7) | (99.7) | (95.9) | (99.0) | (94.4) | (96.7) | (99.8) |
| Redundancy | 20.0 | 79.1 (67.8) | 19.4 | 38.2 | 18.8 | 56.4 | 111 | 9.95 | 100 | 19.9 | 19.7 | 115 | 19.8 |
| | (17.0) | | (18.9) | (35.7) | (15.3) | (39.1) | (25.7) | (9.92) | (34.2) | (20.7) | (18.9) | (29.9) | (17.9) |
| No. of unique | 21,687 | 14,907 | 18,836 | 18,192 | 15,788 | 24,343 | 19,022 | 17,866 | 19,867 | 18,726 | 15,858 | 28,896 | 21,124 |
| reflections | | | | | | | | | | | | | |

Refinement

| | | | | | | | | | |
|---|---|---|---|---|---|---|---|---|---|
| Resolution (Å) | 74.8-2.79 | 75.2- | 75.4- | 74.3- | 99.6-2.71 | 75.1- | 75.2-3.12 | 75.2-2.80 | 75.5- |
| | | 2.95 | 2.99 | 3.08 | | 2.95 | | | 2.85 |
| R_{work}/R_{free} | 0.179/0.204 | 0.174/ | 0.151 / | 0.163/ | 0.180/0.220 | 0.160/ | 0.157/0.179 | 0.167/0.191 | 0.184/ |
| | | 0.172 | 0.181 | 0.191 | | 0.178 | | | 0.212 |

No. Atoms

| | | | | | | | | | |
|---|---|---|---|---|---|---|---|---|---|
| RNA | 2,800 | 2,839 | 2,814 | 2,771 | 2,797 | 2,792 | 2,772 | 2,752 | 2750 |
| Mg²⁺ | 4 | 4 | 4 | 4 | 4 | 3 | 5 | 4 | 4 |
| K⁺ | 1 | 1 | 1 | 1 | 1 | 1 | 1 | 1 | 1 |
| SO₄ | 5 | 5 | 5 | 5 | 5 | 10 | 5 | 5 | 5 |

¹R-factors are defined as: Rₘₑₐₛ = Σₕₖₗ \sqrt{N(hkl)/N(hkl) − 1} × Σᵢ|Iᵢ(hkl) − [symbol] I(hkl) [symbol] |/ΣₕₖₗΣᵢIᵢ(hkl) R_{p.i.m.} = Σₕₖₗ \sqrt{1/N(hkl) − 1} × Σᵢ|Iᵢ(hkl) − [symbol] I(hkl) [symbol] |/ΣₕₖₗΣᵢIᵢ(hkl)

TABLE 2
Thermodynamic parameters for pri-miRNA apical junction and loop folding at 50
mM NaCl, reported as ± standard deviation.

| | Non- | Loop | | | | |
|---|---|---|---|---|---|---|
| RNA | canonical | length | T_{M} | ΔH | ΔS | ΔG_{37° C.} |
| (terminal bp) | pair | (nt) | (° C.) | (kcal/mol) | (e.u.) | (kcal/mol) |
| pri-miR-378a | C-A | 8 | 62.9 ± 0.8 | −42.1 ± 1.6 | −125.4 ± 4.9 | −3.24 ± 0.12 |
| (A-U) | | | | | | |
| pri-miR-340 | U-U | 7 | 64.9 ± 0.5 | −47.7 ± 1.3 | −141.1 ± 3.7 | −3.94 ± 0.12 |
| (U-A) | | | | | | |
| pri-miR-300 | U-U | 7 | 72.4 ± 0.6 | −46.8 ± 4.6 | −135.5 ± 13.4 | −4.79 ± 0.42 |
| (C-G) | | | | | | |
| pri-miR-202 | — | 6 | 69.5 ± 0.3 | −42.1 ± 4.3 | −122.8 ± 12.6 | −3.98 ± 0.38 |
| (G-C) | | | | | | |
| pri-miR-208a | U-A | 5 | 72.2 ± 0.5 | −44.7 ± 1.5 | −129.5 ± 4.4 | −4.56 ± 0.13 |
| (G-C) | | | | | | |
| pri-miR-449c | U-U | 5 | 70.8 ± 0.5 | −40.4 ± 1.0 | −117.5 ± 2.9 | −3.97 ± 0.08 |
| (A-U) | | | | | | |
| pri-miR-320b-2 | — | 4 | 72.3 ± 0.7 | −33.3 ± 1.4 | −96.4 ± 4.1 | −3.40 ± 0.12 |
| (U-A) | | | | | | |
| pri-miR-19b-2 | — | 4 | 70.6 + 1.0 | −30.4 ± 1.2 | −88.5 ± 3.2 | −2.97 ± 0.18 |
| (A-U) | | | | | | |

### DISCLOSURE REFERENCES

1. Ha, M. and V. N. Kim, Regulation of microRNA biogenesis. Nat. Rev. Mol. Cell Biol., 2014. 15: 509-24.
2. Krasilnikov, A. S., et al., Crystal structure of the specificity domain of ribonuclease P. Nature, 2003. 421: 760-4.
3. Reiss, C. W., Y. Xiong, and S. A. Strobel, Structural Basis for Ligand Binding to the Guanidine-I Riboswitch. Structure, 2017. 25: 195-202.
4. Byrne, R. T., et al., The crystal structure of unmodified tRNAPhe from Escherichia coli. Nucleic Acids Res, 2010. 38: 4154-62.
5. Auyeung, V. C., et al., Beyond secondary structure: primary-sequence determinants license pri-miRNA hairpins for processing. Cell, 2013. 152: 844-58.
6. Chen, Y., et al., Rbfox proteins regulate microRNA biogenesis by sequence-specific binding to their precursors and target downstream Dicer. Nucleic Acids Res., 2016. 44: 4381-95.
7. Zeng, Y., R. Yi, and B. R. Cullen, Recognition and cleavage of primary microRNA precursors by the nuclear processing enzyme Drosha. EMBO J, 2005. 24: 138-148.
8. Zhang, X. and Y. Zeng, The terminal loop region controls microRNA processing by Drosha and Dicer. Nucleic Acids Res, 2010. 38: 7689-97.
9. Ma, H., et al., Lower and upper stem-single-stranded RNA junctions together determine the Drosha cleavage site. Proc Natl Acad Sci USA, 2013. 110: 20687-92.
10. Fang, W. and D. P. Bartel, The Menu of Features that Define Primary MicroRNAs and Enable De Novo Design of MicroRNA Genes. Mol. Cell, 2015. 60: 131-45.
11. Shortridge, M. D., et al., A Macrocyclic Peptide Ligand Binds the Oncogenic MicroRNA-21 Precursor and Suppresses Dicer Processing. ACS Chem. Biol., 2017. 12: 1611-1620.
12. Chirayil, S., et al., NMR characterization of an oligonucleotide model of the miR-21 pre-element. PloS One, 2014. 9: e108231.
13. Kozomara, A. and S. Griffiths-Jones, miRBase: integrating microRNA annotation and deep-sequencing data. Nucleic Acids Res., 2011. 39: D152-7.
14. Zuker, M., Mfold web server for nucleic acid folding and hybridization prediction. Nucleic Acids Res, 2003. 31: 3406-3415.
15. Gao, A. and A. Serganov, Structural insights into recognition of c-di-AMP by the ydaO riboswitch. Nat. Chem. Biol., 2014. 10: 787-92.
16. Serra, M. J., T. J. Axenson, and D. H. Turner, A model for the stabilities of RNA hairpins based on a study of the sequence dependence of stability for hairpins of six nucleotides. Biochemistry, 1994. 33: 14289-96.
17. Triboulet, R., et al., Post-transcriptional control of DGCR8 expression by the Microprocessor. RNA, 2009. 15: 1005-11.
18. Kadener, S., et al., Genome-wide identification of targets of the drosha- pasha/DGCR8 complex. RNA, 2009. 15: 537-45.
19. Macias, S., et al., DGCR8 HITS-CLIP reveals novel functions for the Microprocessor. Nat. Struct. Mol. Biol., 2012. 19: 760-766.
20. Heras, S. R., et al., The Microprocessor controls the activity of mammalian retrotransposons. Nat Struct Mol Biol, 2013. 20: 1173-81.
21. Han, J., et al., Posttranscriptional cross regulation between Drosha and DGCR8. Cell, 2009. 136: 75-84.
22. Moon, A. F., et al., A synergistic approach to protein crystallization: combination of a fixed-arm carrier with surface entropy reduction. Protein Sci., 2010. 19: 901-13.
23. Terasaka, N., et al., A human microRNA precursor binding to folic acid discovered by small RNA transcriptomic SELEX. RNA, 2016. 22: 1918-1928.
24. Nguyen, T. A., et al., Functional Anatomy of the Human Microprocessor. Cell, 2015. 161. 1374-87.
25. Quick-Cleveland, J., et al., The DGCR8 RNA-binding heme domain recognizes primary microRNAs by clamping the Hairpin. Cell Rep., 2014. 7: 1994-2005.
26. Michlewski, G., et al., Posttranscriptional regulation of miRNAs harboring conserved terminal loops. Mol Cell, 2008. 32: 383-93.
27. Brakier-Gingras, L., J. Charbonneau, and S. E. Butcher, Targeting frameshifting in the human immunodeficiency virus. Expert Opin. Ther. Targets, 2012. 16: 249-58.
28. Kao, C., M. Zheng, and S. Rudisser, A simple and efficient method to reduce nontemplated nucleotide addition at the 3 termimus of RNAs transcribed by T7 RNA polymerase. RNA, 1999. 5: 1268-72.
29. Kabsch, W., XDS. Acta Crystallogr. D Biol. Crystallogr., 2010. 66: 125-32.
30. Adams, P. D., et al., PHENIX: a comprehensive Python-based system for macromolecular structure solution. Acta Crystallogr D Biol Crystallogr, 2010. 66: 213-21.
31. Emsley, P., et al., Features and development of Coot. Acta Crystallogr. D Biol. Crystallogr., 2010. 66: 486-501.
32. Liebschner, D., et al., Polder maps: improving OMIT maps by excluding bulk solvent. Acta crystallographica. Section D, Structural biology, 2017. 73: 148-157.
33. Cheng, C. Y., F. C. Chou, and R. Das, Modeling complex RNA tertiary folds with Rosetta. Methods Enzymol., 2015. 553: 35-64.
34. Milligan, J. F., et al., Oligoribonucleotide synthesis using T7 RNA polymerase and synthetic DNA templates. Nucleic Acids Res., 1987. 15: 8783-98.
35. Partin, A. C., et al., Heme enables proper positioning of Drosha and DGCR8 on primary microRNAs. Nat. Commun., 2017. 8: 1737.
36. Abraham, M. J., et al., GROMACS: High performance molecular simulations through multi-level parallelism from laptops to supercomputers. SoftwareX, 2015. 1-2: 19-25.
37. Langmead, B. and S. L. Salzberg, Fast gapped-read alignment with Bowtie 2. Nat. Methods, 2012. 9: 357-9.

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

All publications, patents, and patent applications cited herein are hereby incorporated by reference in their entirety for all purposes.

1. A composition of matter comprising a ribonucleic acid having an at least 90% sequence identity to:
GGUUGCCGAAUCCGAAAGGUACGGAGGAACCGCUUUUUGGGGUUAAUC UGCAGUGAAGCUGCAGUAGGGAUACCUUCUGUCCCGCACCCGACAGCU AACUCCGGAGGCAAUAAAGGAAGGAG (SEQ ID NO: 1), wherein: residues 14-17 (GAAA) of the ribonucleic acid are replaced with a heterologous segment of nucleic acids that is between 4 and 33 nucleotides in length.

2. The composition of claim 1, further comprising an agent that binds to the ribonucleic acid.

3. The composition of claim 2, wherein the agent is a polynucleotide that hybridizes to the ribonucleic acid.

4. The composition of claim 1, wherein the heterologous segment of nucleic acids forms a loop structure in a naturally occurring RNA molecule.

5. The composition of claim 4, wherein the heterologous segment of nucleic acids includes the complete loop structure, and optionally between 0-5 base pairs of a stem structure in the naturally occurring RNA molecule.

6. A system/kit for observing RNA structures comprising:
a plasmid comprising a DNA sequence encoding a ribonucleic acid having an at least 90% identity to:
(SEQ ID NO: 1)
GGUUGCCGAAUCCGAAAGGUACGGAGGAACCGCUUUUUGGGGUUAAUC
UGCAGUGAAGCUGCAGUAGGGAUACCUUCUGUCCCGCACCCGACAGCU
AACUCCGGAGGCAAUAAAGGAAGGAG.

7. The system/kit of claim 6, further comprising a promoter for expressing the ribonucleic acid.

8. The system/kit of claim 7, further comprising an RNA polymerase.

9. The system/kit of claim 6, further comprising one or more primers that hybridize to a stretch of nucleic acids in the plasmid.

10. A method of obtaining information on a structure of a ribonucleic acid comprising:
obtaining a ribonucleic acid having an at least 90% identity to SEQ ID NO: 1;
substituting residues corresponding to loop residues 14-17 (GAAA) in SEQ ID NO: 1 with a heterologous segment of nucleic acids that is between 4 and 33 nucleotides in length to so as to form a fusion ribonucleic acid molecule;
crystallizing the fusion ribonucleic acid molecule;
performing an X-ray or electron crystallographic technique on the fusion ribonucleic acid molecule; and
observing the results of the X-ray or electron crystallographic technique such that information on the structure of the heterologous segment of nucleic acids is obtained.

11. The method of claim 10, wherein the fusion ribonucleic acid molecule is combined with an agent that binds to the ribonucleic acid prior to the crystallographic analysis.

12. The method of claim 11, wherein the agent is a polynucleotide that hybridizes to the ribonucleic acid.

13. The method of claim 11, wherein the crystallographic analysis includes a comparison to a control sample lacking the agent that binds to the ribonucleic acid.

14. The method of claim 11, wherein a plurality of fusion ribonucleic acid molecules are combined with a plurality of agents that bind to the ribonucleic acid prior to the X-ray or electron crystallographic technique.

15. The method of claim 14, wherein at least two agents are combined with the fusion ribonucleic acid molecules.

16. A method of performing a crystallographic analysis on a polynucleotide, the method comprising:
(a) selecting a first polynucleotide, wherein the first polynucleotide comprises a polynucleotide sequence of a first miRNA;
(b) identifying a segment of polynucleotides that forms a first loop region in the first miRNA;
(c) selecting a second polynucleotide, wherein the second polynucleotide comprises the polynucleotide sequence of a second miRNA;
(d) identifying a segment of polynucleotides that forms a first loop region in the second miRNA;
(e) forming a fusion polynucleotide constructed so that the segment of polynucleotides comprising the first loop region on the first polynucleotide is substituted with the segment of polynucleotides comprising the first loop region on the second polynucleotide; and
(f) crystallographically analyzing the fusion polynucleotide so as to observe a three dimensional structure of the fusion polynucleotide;
so that a crystallographic analysis of the polynucleotide is performed.

17. The method of claim 16, wherein the first miRNA is a miRNA having at least 90% sequence identity to:
GGUUGCCGAAUCCGAAAGGUACGGAGGAACCGCUUUUUGGGGUUAAUCUGCA GUGAAGCUGCAGUAGGGAUACCUUCUGUCCCGCACCCGACAGCUAACUCCGGA GGCAAUAAAGGAAGGAG (SEQ ID NO: 1), wherein: residues 14-17 (GAAA) of the ribonucleic acid are replaced with a heterologous segment of nucleic acids comprising the first loop region on the second polynucleotide that is between 4 and 33 nucleotides in length.

18. The method of claim 17, wherein:
the first polynucleotide comprises the sequence of SEQ ID NO: 1; and/or
the second miRNA comprises a human miRNA.

19. The method of claim 17, wherein the crystallographic analysis is an X-ray or electron crystallographic technique.

20. The method of claim 17, wherein the crystallographic analysis is performed in the presence of agent that binds to the fusion polynucleotide.


SEQUENCE LISTING (49 sequences)

SEQ ID NO: 1 - RNA, 122 nt, Unknown
  Description of Unknown Naturally occurring ribonucleic acid sequence
gguugccgaauccgaaagguacggaggaaccgcuuuuugggguuaaucugcagugaagcugcaguagggauaccuucugucccgcacccgacagcuaacuccggaggcaauaaaggaaggag

SEQ ID NO: 2 - DNA, 19 bp, Artificial Sequence
  Description of Artificial Sequence Synthetic primer
cgtggatccggtcccattc

SEQ ID NO: 3 - DNA, 54 bp, Artificial Sequence
  Description of Artificial Sequence Synthetic primer
ctataggttgccgaatccgtggtgtggagtctggtacggaggaaccgctttttg

SEQ ID NO: 4 - DNA, 56 bp, Artificial Sequence
  Description of Artificial Sequence Synthetic primer
ctataggttgccgaatccagtggtgtggagtcttggtacggaggaaccgctttttg

SEQ ID NO: 5 - DNA, 58 bp, Artificial Sequence
  Description of Artificial Sequence Synthetic primer
ctataggttgccgaatccgagtggtgtggagtcttcggtacggaggaaccgctttttg

SEQ ID NO: 6 - DNA, 60 bp, Artificial Sequence
  Description of Artificial Sequence Synthetic primer
ctataggttgccgaatccagagtggtgtggagtcttctggtacggaggaaccgctttttg

SEQ ID NO: 7 - DNA, 39 bp, Artificial Sequence
  Description of Artificial Sequence Synthetic primer
gcagaattctaatacgactcactataggttgccgaatcc

SEQ ID NO: 8 - DNA, 20 bp, Artificial Sequence
  Description of Artificial Sequence Synthetic primer
  Description of Combined DNA/RNA Molecule Synthetic oligonucleotide
cuccttcctttattgcctcc

SEQ ID NO: 9 - DNA, 22 bp, Artificial Sequence
  Description of Artificial Sequence Synthetic primer
ggtacggaggaaccgctttttg

SEQ ID NO: 10 - DNA, 46 bp, Artificial Sequence
  Description of Artificial Sequence Synthetic primer
ctataggttgccgaatccatatgtggtacggaggaaccgctttttg

SEQ ID NO: 11 - DNA, 48 bp, Artificial Sequence
  Description of Artificial Sequence Synthetic primer
ctataggttgccgaatccgatctggcggtacggaggaaccgctttttg

SEQ ID NO: 12 - DNA, 47 bp, Artificial Sequence
  Description of Artificial Sequence Synthetic primer
ctataggttgccgaatccgatgctcggtacggaggaaccgctttttg

SEQ ID NO: 13 - DNA, 49 bp, Artificial Sequence
  Description of Artificial Sequence Synthetic primer
ctataggttgccgaatccctttacttgggtacggaggaaccgctttttg

SEQ ID NO: 14 - DNA, 46 bp, Artificial Sequence
  Description of Artificial Sequence Synthetic primer
ctataggttgccgaatccaaagttggtacggaggaaccgctttttg

SEQ ID NO: 15 - DNA, 49 bp, Artificial Sequence
  Description of Artificial Sequence Synthetic primer
ctataggttgccgaatccatgtcgtttggtacggaggaaccgctttttg

SEQ ID NO: 16 - DNA, 50 bp, Artificial Sequence
  Description of Artificial Sequence Synthetic primer
ctataggttgccgaatccacctagaaatggtacggaggaaccgctttttg

SEQ ID NO: 17 - DNA, 47 bp, Artificial Sequence
  Description of Artificial Sequence Synthetic primer
ctataggttgccgaatccatgatttggtacggaggaaccgctttttg

SEQ ID NO: 18 - DNA, 39 bp, Artificial Sequence; identical to SEQ ID NO: 7
  Description of Artificial Sequence Synthetic primer

SEQ ID NO: 19 - DNA, 33 bp, Artificial Sequence
  Description of Artificial Sequence Synthetic oligonucleotide
ggaacacatatgttcctatagtgagtcgtatta

SEQ ID NO: 20 - DNA, 35 bp, Artificial Sequence
  Description of Artificial Sequence Synthetic oligonucleotide
ggaacgccagatcgttcctatagtgagtcgtatta

SEQ ID NO: 21 - DNA, 34 bp, Artificial Sequence
  Description of Artificial Sequence Synthetic oligonucleotide
ggaacgagcatcgttcctatagtgagtcgtatta

SEQ ID NO: 22 - DNA, 36 bp, Artificial Sequence
  Description of Artificial Sequence Synthetic oligonucleotide
ggaaccaagtaaaggttcctatagtgagtcgtatta

SEQ ID NO: 23 - DNA, 33 bp, Artificial Sequence
  Description of Artificial Sequence Synthetic oligonucleotide
ggaacaactttgttcctatagtgagtcgtatta

SEQ ID NO: 24 - DNA, 36 bp, Artificial Sequence
  Description of Artificial Sequence Synthetic oligonucleotide
ggaacaaacgacatgttcctatagtgagtcgtatta

SEQ ID NO: 25 - DNA, 37 bp, Artificial Sequence
  Description of Artificial Sequence Synthetic oligonucleotide
ggaacatttctaggtgttcctatagtgagtcgtatta

SEQ ID NO: 26 - DNA, 34 bp, Artificial Sequence
  Description of Artificial Sequence Synthetic oligonucleotide
ggaacaaatcatgttcctatagtgagtcgtatta

SEQ ID NO: 27 - DNA, 13 bp, Unknown
  Description of Unknown Naturally occurring ribonucleic acid sequence
ggttgccgaatcc

SEQ ID NO: 28 - DNA, 105 bp, Unknown
  Description of Unknown Naturally occurring ribonucleic acid sequence
ggtacggaggaaccgctttttggggttaatctgcagtgaagctgcagtagggataccttctgtcccgcacccgacagctaactccggaggcaataaaggaaggag

SEQ ID NO: 29 - RNA, 151 nt, Artificial Sequence
  Description of Artificial Sequence Synthetic polynucleotide
  modified_base (14)..(46): a, c, u, or g
  misc_feature (14)..(46): This region may encompass 4-33 nucleotides
gguugccgaauccnnnnnnnnnnnnnnnnnnnnnnnnnnnnnnnnngguacggaggaaccgcuuuuugggguuaaucugcagugaagcugcaguagggauaccuucugucccgcacccgacagcuaacuccggaggcaauaaaggaaggag

SEQ ID NO: 30 - RNA, 118 nt, Artificial Sequence
  Description of Artificial Sequence Synthetic polynucleotide
gguugccgaauccgguacggaggaaccgcuuuuugggguuaaucugcagugaagcugcaguagggauaccuucugucccgcacccgacagcuaacuccggaggcaauaaaggaaggag

SEQ ID NO: 31 - RNA, 20 nt, Unknown
  Description of Unknown Naturally occurring ribonucleic acid sequence
ggaacaccuagaaauguucc

SEQ ID NO: 32 - RNA, 19 nt, Unknown
  Description of Unknown Naturally occurring ribonucleic acid sequence
ggaacuuugcuguaguucc

SEQ ID NO: 33 - RNA, 19 nt, Unknown
  Description of Unknown Naturally occurring ribonucleic acid sequence
ggaaccuuuacuugguucc

SEQ ID NO: 34 - RNA, 18 nt, Unknown
  Description of Unknown Naturally occurring ribonucleic acid sequence
ggaacgaucuggcguucc

SEQ ID NO: 35 - RNA, 18 nt, Unknown
  Description of Unknown Naturally occurring ribonucleic acid sequence
ggaacgaucuggcguucc

SEQ ID NO: 36 - RNA, 16 nt, Unknown
  Description of Unknown Naturally occurring ribonucleic acid sequence
ggaacaugauuguucc

SEQ ID NO: 37 - RNA, 16 nt, Unknown
  Description of Unknown Naturally occurring ribonucleic acid sequence
ggaacuugaaaguucc

SEQ ID NO: 38 - RNA, 16 nt, Unknown
  Description of Unknown Naturally occurring ribonucleic acid sequence
ggaacauauguguucc

SEQ ID NO: 39 - RNA, 55 nt, Unknown
  Description of Unknown Naturally occurring ribonucleic acid sequence
ggugacuccagguccuguguguuaccuagaaauaagcacuggacuuggagucacc

SEQ ID NO: 40 - RNA, 40 nt, Unknown
  Description of Unknown Naturally occurring ribonucleic acid sequence
ggagacugauugucauaugucguuugugggauccgucucc

SEQ ID NO: 41 - RNA, 39 nt, Unknown
  Description of Unknown Naturally occurring ribonucleic acid sequence
gguucacgcauuugcuuuacuugcaaugauuauacaacc

SEQ ID NO: 42 - RNA, 52 nt, Unknown
  Description of Unknown Naturally occurring ribonucleic acid sequence
gguaugcauauacuucuuugaggaucuggccuaaagagguauagggcaugcc

SEQ ID NO: 43 - RNA, 39 nt, Unknown
  Description of Unknown Naturally occurring ribonucleic acid sequence
ggcccggguuauaccugaugcucacguauaagacgagcc

SEQ ID NO: 44 - RNA, 54 nt, Unknown
  Description of Unknown Naturally occurring ribonucleic acid sequence
ggcaguguauugcuagcggcuguuaaugauuuuaacaguugcuaguugcacucc

SEQ ID NO: 45 - RNA, 47 nt, Unknown
  Description of Unknown Naturally occurring ribonucleic acid sequence
ggucucuucccagauuucccaaaguugggaaaagcuggguugagacc

SEQ ID NO: 46 - RNA, 45 nt, Unknown
  Description of Unknown Naturally occurring ribonucleic acid sequence
gguuugcauuucagcguauauauguauauguggcugugcaaaucc

SEQ ID NO: 47 - RNA, 58 nt, Unknown
  Description of Unknown Naturally occurring ribonucleic acid sequence
  modified_base (1)..(1): a, c, u, g, unknown or other
  modified_base (28)..(28): a, c, u, g, unknown or other
  modified_base (31)..(33): a, c, u, g, unknown or other
nguguauuugacaarcugacacucyrunurnnngagugucaguuugucaaauacccca

SEQ ID NO: 48 - RNA, 52 nt, Unknown
  Description of Unknown Naturally occurring ribonucleic acid sequence
kduygcccccagcccgurcucuguvcgacacuauwuagagagggggcgrcmk

SEQ ID NO: 49 - RNA, 54 nt, Unknown
  Description of Unknown Naturally occurring ribonucleic acid sequence
auugccaggacgaccggguccuuucuuggauuaacccgcucaaugccuggagau